(19) United States
(12) Patent Application Publication
(10) Pub. No.: US 2024/0027430 A1
(43) Pub. Date: Jan. 25, 2024

(54) Cell and Other Bio-Entity Identification and Counting

(71) Applicant: Essenlix Corporation, Monmouth Junction, NJ (US)

(72) Inventors: Stephen Y. CHOU, Princeton, NJ (US); Yu SUN, Basking Ridge, NJ (US); Wei DING, Princeton, NJ (US); Ji Qi, Hillsborough, NJ (US); Mingquan WU, Princeton Junction, NJ (US); Shengjian CAI, Bridgewater, NJ (US); Wu CHOU, Basking Ridge, NJ (US)

(73) Assignee: Essenlix Corporation, Monmouth Junction, NJ (US)

(21) Appl. No.: 18/373,267

(22) Filed: Sep. 26, 2023

Related U.S. Application Data
(63) Continuation of application No. PCT/US22/22229, filed on Mar. 28, 2022.
(60) Provisional application No. 63/166,933, filed on Mar. 26, 2021.
(60) Provisional application No. 63/166,934, filed on Mar. 26, 2021.

(51) Int. Cl. G01N 33/50; G01N 1/30; G01N 21/64; G06V 20/69

(52) U.S. Cl. CPC G01N 33/5094; G01N 1/30; G01N 21/6456; G01N 21/6428; G06V 20/693; G06V 20/698; G01N 2001/302; G01N 2021/6439

(57) ABSTRACT

The disclosure provides a method for identifying a bio-entity including a cell type and count in a sample. The method includes: providing a device comprising a first plate, a second plate, and a patterned structural element; depositing the sample between the first and second plates; reducing the spacing of the first and second plates so that the first and second plates are in a closed configuration to compress the sample into a layer; and imaging the sample to obtain an image; and measuring and analyzing the image against a database generated with a machine learning model to obtain the bio-entity of the sample. The sample can be a blood sample, and the method can be a white blood cell differential test conducted with a mobile phone.

### CROSS REFERENCE TO RELATED APPLICATION

This application is a by-pass continuation of PCT/US22/22229, filed on Mar. 28, 2022, which claims priority to the US provisional applications with serial nos. 63/166,933 and 63/166,934, filed Mar. 26, 2021, the entire contents of which are incorporated herein by reference.

### FIELD

The embodiments described herein generally pertain to the identification of a sample. Specifically, the embodiments relate to a device, a system, and a method for identifying a bio sample, including cell count and cell differential.

### BACKGROUND

White blood cells, also called leukocytes or leucocytes, are the immune system cells that are involved in protecting the body against, for example, infectious diseases and foreign invaders. The white blood cells are typically produced and derived from multipotent cells in the bone marrow known as hematopoietic stem cells.

### SUMMARY

The disclosure provides a method for identifying a cell in a sample, the method comprising:

obtaining two plates; sandwiching the sample between two plates;

reducing the spacing of the two plates to a dimension that is less than the dimension of the cell that is not compressed by the plates;

imaging by taking one or more images of the cell between the two plates; and identifying the cell by analyzing the images.

In some embodiments, the method is used to identify another bio-entity instead of a cell.

In some embodiments, the spacing between the two plates is reduced to a dimension insofar as the cell forms a monolayer between the two plates.

In some embodiments, one of the images is a bright-field image, and one of the images is a dark-field image.

In some embodiments, one of the images is a bright-field image, and one of the images is a fluorescence image.

In some embodiments, one of the images is a bright-field image, one of the images is a dark-field image, and one of the images is a fluorescence image.

In some embodiments, the cell or other bio-entity is deformed by 1%, 5%, 10%, 20%, 30%, 40%, 50%, 60%, 70%, 80%, 90%, or a value between any two of the values.

In some embodiments, the cell or other bio-entity is stained. In some embodiments, the stain is acridine orange (AO). In some embodiments, the stain uses a fluorescence dye and a surfactant together.

In some embodiments, one of the images is a bright-field image taken by a white light, and one of the images is a fluorescence image in the wavelength range from 480 nm or longer.

In some embodiments, one of the images is a bright-field image taken by a white light, and one of the images is a fluorescence-field image in the wavelength range from 500 nm or longer.

In some embodiments, the bright-field image comprises the image of the phenotype of a substructure of the cell or other bio-entity.

In some embodiments, the phenotype comprises the morphology, spectrum, and intensity, and their distribution in the cell or other bio-entity.

In some embodiments, the cell comprises neutrophils, lymphocytes, monocytes, eosinophils and/or basophils.

In some embodiments, the stain concentration is increased to a value that the phenotype of a substructure of the cell or other bio-entity becomes visible in the bright-field image.

In some embodiments, the cell or other bio-entity is stained together with a surfactant, and the stain concentration and/or the surfactant concentration is increased to a value that the phenotype of a substructure of the cell or other bio-entity become visible in the bright-field image.

In some embodiments, for observing and analyzing 3 or 5 differentials of white blood cells (WBC) such as neutrophils (NEU), lymphocytes (LYM), monocytes (MON), eosinophils (EOS), and basophils (BAS), the gap between the two plates has a gap distance of 2 μm to 10 μm. In some embodiments, the gap is preferable 5 μm for observing and analyzing 3 or 5 differentials of white blood cells. In some embodiments, for observing and analyzing the total WBC, the gap between the two plates has a gap distance of 20 μm and 40 μm. In some embodiments the gap is preferable 30 μm for observing and analyzing the total WBC. In some embodiments, the two plates can have two gap distances: one for differentials and the other is for total WBC.

In some embodiments, one or both plates has multiple heights, so that the gap in one area is 2 μm to 10 μm for analyzing neutrophils (NEU), lymphocytes (LYM), monocytes (MON), eosinophils (EOS), and basophils (BAS); and the gap in another area of the plate is 20 μm and 40 μm for analyzing total WBC.

In some embodiments, one or both plates has multiple heights, so that the gap in one area is 2 μm to 10 μm for analyzing neutrophils, lymphocytes, monocytes, eosinophils, and basophils; and the gap in another area of the plate is 20 μm and 40 μm for analyzing total WBC.

In some embodiments, one or both plates has multiple heights, so that the gap in one area is 5 μm for neutrophils, lymphocytes, monocytes, eosinophils, and basophils; and the gap in another area of the plate is 20 μm and 40 μm for analyzing total WBC.

In some embodiments, one or both plates has multiple heights, so that the gap in one area is 2 μm to 10 μm and the gap in another area of the plate is 20 μm and 40 μm.

In some embodiments, one or both plates has multiple heights, so that the gap in one area is 5 μm and the gap in another area of the plate is 30 μm.

In some embodiments, the whole blood sample is analyzed without dilution.

### BRIEF DESCRIPTION OF THE DRAWINGS

The drawings described herein are for illustration purposes only and are not intended to limit the scope of the disclosure. Portions or elements of a drawing may not necessarily be in the same scale in the same drawing or across the drawings. A portion or element of a drawing may be shown exaggerated or enlarged to provide a detailed view of the portion or element. A portion or element of a drawing may also be enlarged when illustrated in the other drawing(s) for a detailed view. Reference may be made to the accompanying drawings that form a part of this disclosure and which illustrate embodiments described herein. Like references generally refer to like features.

FIG. 1 schematically illustrates a Q-Card in accordance with some embodiments.

FIG. 2 schematically illustrates a deformation of a white blood cell in a Q-Card by changing the spacing of the Q-Card in accordance with one or more embodiments. (a) schematically shows that the white blood cell is not deformed in the Q-Card if the spacing is larger than the size of the white blood cell. (b) schematically shows that the white blood cell is deformed and enlarged in the Q-Card when the spacing is less than the size of the white blood cell.

FIG. 3 schematically illustrates an imaging optics system in a fluorescent mode for capturing a fluorescent image of a sample in accordance with one or more embodiments.

FIG. 4 schematically illustrates an imaging optics system in a bright-field mode for capturing a bright-field image of a sample in accordance with one or more embodiments.

FIG. 5 schematically illustrates a mechanical mechanism of sliding in and out the filter between lenses, in accordance with one or more embodiments.

FIG. 6 shows microscopic images of acridine orange-stained white blood cells under bright and fluorescence fields, in accordance with some embodiments.

FIG. 7 shows iMOST images of acridine orange-stained white blood cells under bright and fluorescence fields, in accordance with some embodiments.

FIG. 8 schematically illustrates a sample patch construction, in accordance with some embodiments.

FIG. 9 schematically illustrates a cell image patch construction using an existing WBC detection model, in accordance with some embodiments.

FIG. 10 schematically illustrates a machine learning network architecture, in accordance with some embodiments.

FIG. 11 schematically illustrates an inference pipeline for cell differential, in accordance with some embodiments.

### DETAILED DESCRIPTION

The following detailed description illustrates certain embodiments of the invention by way of example and not by way of limitation. Any section headings and subtitles used herein are for organizational purposes only and are not to be construed as limiting the subject matter described in any way. The contents under a section heading and/or subtitle are not limited to the section heading and/or subtitle, but apply to the entire disclosure.

The term “a,” “an,” or “the” cover both the singular and the plural reference, unless the context clearly dictates otherwise. The terms “comprise,” “have,” “include,” and “contain” are open-ended terms, which means “include but not limited to,” unless otherwise indicated.

A white blood cell differential (WBC-Diff) generally means a medical test that provides information about the types and amounts of each type of white blood cells such as neutrophils (NEU), lymphocytes (LYM), monocytes (MON), eosinophils (EOS), and basophils (BAS) in a subject's blood. In some embodiments, the WBC-Diff can additionally measure abnormal cell types if they are present. In some embodiments, the measured results are reported as percentages and absolute values, and compared against reference ranges to determine whether the values are normal, low, or high. Changes in the amounts and particular types of white blood cells can aid in the diagnosis of many health conditions, including viral, bacterial, and parasitic infections and blood disorders such as leukemia.

The terms “Q-Card,” “QMAX-device,” “CROF Card (or card),” “COF Card,” “QMAX-Card,” “CROF device,” “COF device,” “CROF plates,” “COF plates,” and “QMAX-plates” are interchangeable and refer to a device that comprises a first plate and a second plate that are movable relative to each other, which forms different configurations, including an open configuration and a closed configuration. The device may or may not comprise spacers that regulate the spacing between the first and the second plates.

The terms “first plate” or “second plate” are plates used in, for example, a Q-Card described herein.

The term “plate” refers to, unless indicated otherwise, one of the first and second plates used in, for example, a Q-Card, which is solid and has a surface that can be used, together with another plate, to compress a sample placed therebetween to reduce a thickness of a sample.

The term “plates” or “two plates” refers to the first and second plates used in, for example, a Q-Card.

The term “the plates are facing each other” refers to a configuration of the first and second plates where the first and second plates at least partially face each other.

The term “spacers” refers to, unless indicated otherwise, mechanical objects that can set a limit on the minimum spacing between the two plates when the spacers are disposed between the two plates and when the two plates are compressed against each other. Namely, in the compressing, the spacers can stop the relative movement of the two plates to prevent the spacing from becoming less than a preset (i.e., predetermined) value. The types of spacers can include an open spacer and an enclosed spacer. The term “open spacer” has a shape that allows a liquid sample to flow around the entire perimeter of the spacer and flow past the spacer. In some embodiments, a pillar is an open spacer. The term “enclosed spacer” has a closed shape that prevents a liquid sample from overflowing the entire perimeter of the spacer and flowing past the perimeter of the spacer. For example, a ring-shaped spacer is an enclosed spacer because it has a ring as a perimeter for holding a liquid sample inside the ring and preventing the liquid sample from flowing outside the ring.

The term “open configuration” described herein means a configuration in which the first and second plates are either partially or completely separate apart, and the spacing between the first and second plates is not regulated by the spacers.

The term “closed configuration” means a configuration in which the first and second plates face each other and stack on each other. In some embodiments, the closed configuration enables the spacers and a relevant volume of a sample to be sandwiched between the two plates, and thereby the thickness of the relevant volume of the sample is regulated by the two plates and the spacers, in which the relevant volume is at least a portion of the entire volume of the sample.

The “inter-spacer distance” means the closest distance between two spacers of the same plate.

The “substantially uniform thickness” means a thickness that is constant or only fluctuates around a mean value, for example, by no more than 10%, and preferably no more than 5%.

The term “iMOST” represents a proprietary instant mobile phone health testing platform developed and manufactured by Essenlix Corporation. The iMOST can measure various biomarkers including biological or chemical health indicators such as, e.g., protein, cells, small molecules, etc., in a single drop of body fluid such as, e.g., blood, urine, saliva, sweat, etc., and converts the results into a digital signal within about 60 seconds using a mobile phone. iMOST can produce test results with a lab-quality accuracy at a low cost with easy operation anytime and anywhere (at home, POC (point-of-care), clinics, hospitals, etc.

The term “and/or” means any one or more of the items in the list joined by “and/or.” As an example, “x and/or y” means any element of the three-element set {(x), (y), (x, y)}. In other words, “x and/or y” means “one or both of x and y.” As another example, “x, y, and/or z” means any element of the seven-element set {(x), (y), (z), (x, y), (x, z), (y, z), (x, y, z)}. In other words, “x, y, and/or z” means “one or more of x, y, and z.”

The embodiments described herein generally pertains to the identification of a sample. Specifically, the embodiments described herein relate to a device and a method for identifying a biological sample, including cell count and cell differential.

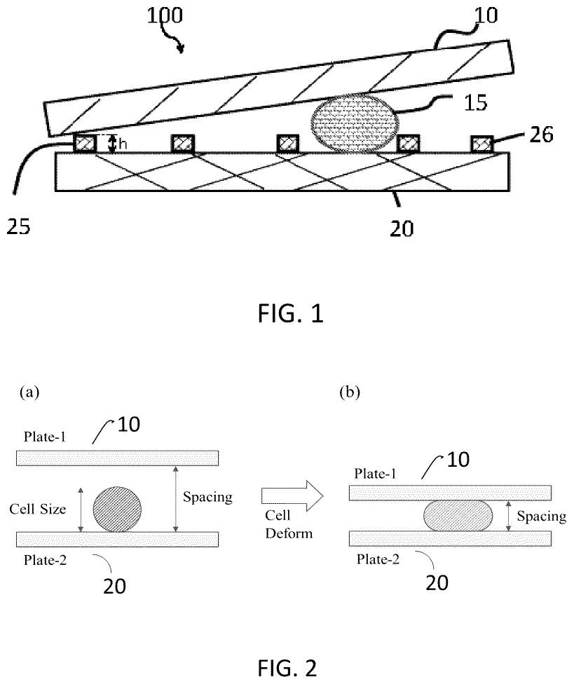

In some embodiments, the disclosure herein provides a device, a system, and a method useful for a blood differential test such as a white blood cell differential test. In some embodiments, the device is a Q-Card. In some embodiments, the system is an iMOST. FIG. 1 schematically illustrates a Q-Card device 100 for analyzing white blood cells (WBC) and white blood cell differential (WBC-Diff) in a sample. In some embodiments, the Q-Card 100 can comprise two plates, including a first plate 10 and a second plate 20. The first plate 10 and the second plate 20 can be movable relative to each other, which forms different configurations. In some embodiments, the second plate 20 is movable relative to the first plate 10 into different configurations, including an open configuration and a closed configuration.

In some embodiments of the open configuration, the first plate 10 and the second plate 20 are at least partially separated from each other, and at least one of the first plate 10 and the second plate 20 receives deposition of sample 15 containing WBC. In some embodiments of the closed configuration, at least a portion of the deposited sample is compressed into a layer of substantially uniform thickness in contact with the first plate and the second plate to form a sample with a substantially uniform thickness. In some embodiments, the layer is a monolayer of cells between the two plates.

In some embodiments, at least one of the two plates has a structural element 25. In some embodiments, the structural element 25 comprises a plurality of spacers 26 affixed thereon. In some embodiments, the first plate 10 has a plurality of spacers affixed thereon. In some embodiments, the second plate 20 has a plurality of spacers affixed thereon. In some embodiments, each of the first plate 10 and the second plate 20 has a plurality of spacers affixed thereon.

In some embodiments, at least one of the two plates has a storage site for storing, for example, a chemical reagent. In some embodiments, the first plate 10 has a storage site. In some embodiments, the second plate 20 has a storage site. In some embodiments, each of the first plate 10 and the second plate 20 has a storage site. In some embodiments, the storage site is coated with at least one reagent.

In some embodiments, the spacing height, which is indicated with the distance “h” in FIG. 1, of the spacers is 1 μm, 2 μm, 3 μm, 5 μm, 10 μm, 20 μm, 30 μm, 50 μm, 100 μm, 150 μm or in a range between any two of the above-mentioned spacing heights.

In some embodiments, the preferred spacing height of the spacers is 2 μm, 3 μm, 5 μm, 10 μm, 15 μm, 30 μm or in a range between any two of the above-mentioned preferred spacing heights.

In some embodiments, the inter-spacer distance is 10 μm, 30 μm, 50 μm, 80 μm, 100 μm, 200 μm, 500 μm, 1000 μm or in a range between any two of the above-mentioned inter-spacer distances.

In some embodiments, the preferred inter-spacer distance is 50 μm, 80 μm, 100 μm, 150 μm, 200 μm, or in a range between any two of the above-mentioned preferred inter-spacer distances.

In some embodiments, the spacer size is 1 μm, 5 μm, 10 μm, 30 μm, 50 μm, 80 μm, 100 μm, 200 μm, 500 μm, or in a range between any two of the above-mentioned space sizes.

In some embodiments, the preferred spacer size is 5 μm, 10 μm, 30 μm, 40 μm, 50 μm, 80 μm, or in a range between any two of the above-mentioned preferred spacer sizes.

### Measuring WBC and WBC-Diff Using a Q-Card

In some embodiments, the spacer height, the spacing between the plates, and/or sample thickness is 5 μm.

In some embodiments, the spacers have a rectangle shape with round corners on the first plate having a thickness of 175 μm. In some embodiments, the second plate is flat with a thickness of 1 mm. In some embodiments, both plates are made of poly(methyl methacrylate).

In some embodiments, the lateral dimension of a spacer is 30 μm by 40 μm. In some embodiments, the round corner of the spacer has a radius of 10 μm. In some embodiments, the spacer is in a rectangular lattice array. In some embodiments, the inter-spacer spacing of spacers is 80 μm. In some embodiments, the period of spacers is 110 μm by 120 μm.

In some embodiments, the reagent is coated by droplet printing into an array. In some embodiments, the reagent is coated onto the first plate. In some embodiments, the reagent is coated by guided flow coating using the spacer on the first plate.

In some embodiments, the reagent comprises a staining agent. In some embodiments, the staining reagent is or comprises acridine orange. In some embodiments, the reagent comprises a cell separation reagent. In some embodiments, the reagent comprises a staining reagent and a cell separation reagent. In some embodiments, the cell separation reagent is a detergent. In some embodiments, the detergent is or comprises a Zwittergent.

In some embodiments, the acridine orange is coated on a plate with an area concentration of 2 to 20 ng/mm². In some embodiments, Zwittergent is coated on a plate with an area concentration of 3 to 30 ng/mm².

In some embodiments, the reagent contains a nucleic acid staining dye such as, for example, YOYO and Hoechst stain.

In some embodiments, the measurement area on the Q-Card is 1 mm² to 100 mm².

In some embodiments, the WBC-Diff includes parameters of neutrophils, lymphocytes, monocytes, eosinophils, and basophils, as well as abnormal cell types if they are present, including but not limited to band neutrophil, immature granulocyte, blast cell, and others.

### Method

In some embodiments, a method of analyzing white blood cell (WBC) and white blood cell differential (WBC-Diff) using a Q-Card or QMAX device, comprising:
(a) providing a Q-Card or QMAX device in the open configuration;
(b) dropping the sample containing WBC inside the device and closing the Q-Card or QMAX device;
(c) imaging the Q-Card or QMAX device in an area containing WBC to obtain a bright-field image and/or fluorescence image;
(b) measuring and analyzing signals from the area containing WBC;
(e) counting and identifying the WBC and WBC subtypes with a trained algorithm.

In some embodiments, a method of analyzing white blood cell (WBC) and white blood cell differential (WBC-Diff) using a Q-Card or QMAX device, comprising:
(a) providing a Q-Card or QMAX device in the close configuration;
(b) dropping a sample containing WBC at an edge of the Q-Card or QMAX device and waiting the sample sucked into an imaging area thereof;
(c) imaging the imaging area containing WBC of the Q-Card or QMAX device to obtain a bright-field image and/or fluorescence image;
(b) measuring and analyzing the signals from the imaging area containing WBC;
(e) counting and identifying the WBC and WBC subtypes with a trained algorithm.

In some embodiments, the sample containing WBC is a whole blood sample. In some embodiments, the sample containing WBC is a whole blood sample without dilution. In some embodiments, the sample containing WBC is capillary or venous whole blood sample with or without anti-agglutination as EDTA.

In some embodiments, the imaging step or process includes but is not limited to bright-field imaging with a broadband light (over 100 nm bandwidth), bright field imaging with a narrow band light (less than 100 nm bandwidth), fluorescence imaging.

In some embodiments, the fluorescence imaging is conducted with an excitation light at 450 nm to 500 nm and has an emission light at 500 nm to 550 nm and 600 nm to 700 nm.

In some embodiments, the fluorescence imaging is conducted with an excitation light at 475 nm and has an emission light at both 525 nm and 650 nm.

### Cell Deformation

In some embodiments, the white blood cells are pressed and deformed in the Q-Card or QMAX device to facilitate the imaging and analysis, as shown in FIG. 2.

In some embodiments, the white blood cells are pressed to a layer having a thickness of 1 μm, 2 μm, 3 μm, 5 μm, 10 μm, 15 μm or in a range between any two of the thicknesses.

In some embodiments, the white blood cells are pressed to a layer having a preferred thickness of 2 μm, 3 μm, 5 μm, 10 μm, or in a range between any two of the preferred thicknesses.

FIG. 2 schematically illustrates a deformation of a white blood cell in a Q-Card by changing the spacing between the first plate 10 and the second plate 20 of the Q-Card in accordance with some embodiments. FIG. 1(a) schematically shows that the white blood cells are not deformed by the first plate 10 and the second plate 20 of the Q-Card when the spacing between the first plate 10 and the second plate 20 is larger than the size of the white blood cells. FIG. 1(b) schematically shows that the white blood cells are deformed and enlarged or flattened by the first plate 10 and the second plate 20 of the Q-Card when the spacing between the first plate 10 and the second plate 20 is less than the size of the white blood cells. The white blood cells can be deformed in the Q-Card by controlling the spacing between the first plate 10 and the second plate 20.

In some embodiments, the white blood cells are pressed to achieve an area increase of 10%, 20%, 30%, 50%, 80%, 100%, 200%, 300%, 500%, 1000%, or in a range between any two of the values.

In some embodiments, the white blood cells are pressed to achieve a preferred area increase of 50%, 80%, 100%, 200%, 300%, or in a range between any two of the values.

### Optical Imaging System

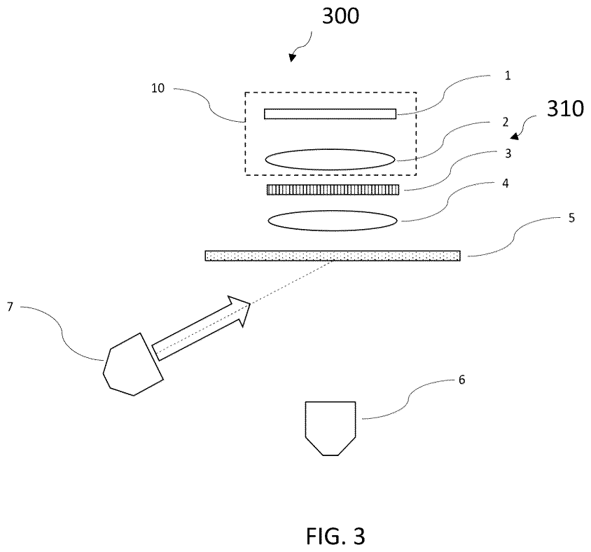

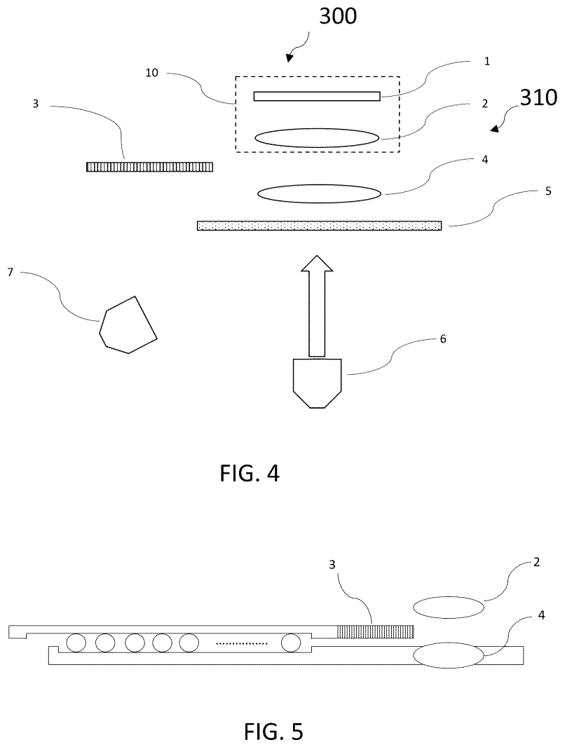

An optical imaging system is developed to capture a bright-field image and a fluorescent image of the same sample area at nearly the same time. FIGS. 3 and 4 schematically illustrate an optical imaging system 300 in fluorescence-field mode and a bright-field mode, respectively. In some embodiments, the imaging optics system comprises a camera module 310, a light source 6, a light source 7, a filter 3, and a lens 4. Camera module 310 comprises an imaging sensor 1 and an internal lens 2. The external lens 4 is placed right below the camera module 310 aligned with its optical axis. The filter 3 is disposed between the internal lens 2 and the external lens 4. In some embodiments, is the filter 3 is a slidable filter. When the filter 3 was inserted between the lens 2 and the lens 4, only a selected wavelength range of light may pass through the lens 2. When filter 3 moves out of the space between the lens 2 and the lens 4, there is no blocking of the wavelength range of the light passing through the lens 2.

In some embodiments, a Q-card 5 can be disposed below or right below the lens 4. The Q-card 5 holds the sample to be tested. In some embodiments, the Q-card 5 is movable in the optical imaging system to expose different areas of the sample under the lens 4. In some embodiments, the Q-card 5 is mechanically movable.

In some embodiments, the light source 6 and the light source 7 are disposed below the Q-card 5, viewed from the orientation of the imaging optics system shown in FIGS. 3 and 4. In some embodiments, the light source 7 is used for the fluorescent excitation and emits a selected wavelength range of light. In some embodiments, the light source 7 illuminates the sample from its bottom side at a particular oblique angle, viewed from the orientation of the imaging optics system shown in FIGS. 3 and 4. In some embodiments, the oblique angle is chosen to be larger than the collecting angle of the lens 4 to minimize the excitation light entering lens 4.

In some embodiments, the light source 6 is used for the bright-field illumination and emits a broadband white light. In some embodiments, light source 6 illuminates the sample from its backside at the normal direction, viewed from the orientation of the imaging optics system shown in FIGS. 3 and 4.

FIG. 3 schematically illustrates a fluorescent mode of the imaging optics system for capturing a fluorescent image of the sample. In some embodiments of the fluorescent mode, the light source 7 is turned on, and the light source 6 is turned off. In some embodiments, the filter 3 is inserted between the lens 4 and the lens 2.

FIG. 4 schematically illustrates a bright-field mode of the imaging optics system for capturing a bright-field image of the sample. In the bright-filed mode, the light source 7 is turned off, and the light source 6 is turned on. in some embodiments, the filter 3 is inserted between the lens 4 and the lens 2.

In some embodiments, the distance between the Q-card 5 and the image sensor 1 is smaller than 20 mm, preferably smaller than 10 mm.

In some embodiments, the distance between the external lens 4 and the internal lens 2 is smaller than 5 mm, preferably smaller than 2 mm.

In some embodiments, the thickness of the filter 3 located between the external lens 4 and the internal lens 2 is smaller than 2 mm. In some embodiments, the thickness is preferably 0.5 mm.

FIG. 5 illustrates some embodiments of a sliding mechanism of the sliding filter 3. In some embodiments, the sliding mechanism enables the sliding filter 3 to slide in and out of space between the lens 2 and the lens 4. In some embodiments, the sliding mechanism utilizes a ball bearing to reduce friction and to extend its lifetime. In some embodiments, there is a groove 34 on a holding structure 32 of the filter 3. In some embodiments, there is another groove 44 on a holding structure 42 of the lens 4. In some embodiments, the sum of the groove depths of these two grooves is less than the diameter of the ball bearing so that there is a small space between the holding structure 32 and the holding structure 42 to avoid friction.

In some embodiments, the ball bearing diameter is 0.5 mm, 1 mm, 1.5 mm, 2 mm or in a range between any two of the above-mentioned diameters.

In some embodiments, the difference of the sum of the groove depth and the ball bearing diameter is 0.1 mm, 0.2 mm, 0.5 mm, 1 mm, or in a range between any two of the above-mentioned differences.

In some embodiments, a method of capturing a pair of bright-field and fluorescent images of a sample area nearly at the same time comprises:

Step 1: mechanically move a Q-card to expose an area of interest of the sample under the imaging lens 4.

Step 2: turn on light source 7 and insert filter 3 between lens 2 and lens 4.

Step 3: capture a fluorescent image of the sample.

Step 4: immediately turn off the light source 7, and then turn on light source 6 and move filter 3 out of the space between lens 2 and lens 4.

Step 5: capture a bright-field image of the sample.

In some embodiments, when taking the bright-field images, a bandpass filter of a given wavelength band can be switched into the space between lens 2 and lens 4. Hence, the absorption of cells at a given wavelength can be measured on the taken image by dividing the pixel intensity of the cell by the intensity of the spacer area.

### Reagent and Coating

In some embodiments, the blood sample is stained with Acridine Orange that has two unique chemical features: 1) intracellular density and distribution dependent on intracellular pH level; 2) nucleic acid-selective fluorescent dye.

Under bright field, acridine orange-stained white blood cells show a colorimetric difference that corresponds to their intracellular pH level, in accordance with some embodiments. The inventors found that acridine orange can form brown color granules when intracellular pH is acidic. For example, acridine orange forms fluorescence yellowish/brownish aggregates/granules under the bright field in the cytoplasm of live eosinophils; however, there is very faint or colorless staining in the cytoplasm of live basophils.

Under the fluorescence field, when acridine orange is excited by blue light (475 nm), it can stain mammalian cell nuclei green (525 nm) and cytoplasm red (650 nm), in accordance with some embodiments. A plurality of images of the blood sample can be acquired. A first stained area, which is disposed within white blood cell candidates, is being identified using a bright-field image. A second stained area, composed of multiple structures including cytoplasm and nuclei within white blood cell candidate, is being identified using a fluorescent (fluorescence field) image. A white blood cell is determined by structural and color features of the stained areas, which satisfy predetermined criteria associated with a white blood cell.

In some embodiments, 5 μl to 10 μl of a reagent containing acridine orange (AO) and zwittergent 3-14 are pre-coated on 5 μm spacing Q-card with a size of 10 mm to 20 mm.

In some embodiments, the reagent contains 0.6 mg/ml of and 1.2 mg/ml of Zwittergent 3-14 for microscopic detection.

In some embodiments, the reagent contains 0.9 mg/ml of acridine orange and 1 mg/ml of Zwittergent 3-14 for iMOST system detection.

In some embodiments, the reagent contains 0.6 mg/ml of acridine orange and 1.9 mg/ml of Zwittergent 3-14 for iMOST system detection.

In some embodiments, the reagent contains 0.6 mg/ml of acridine orange and 2 mg/ml of Zwittergent 3-14 for iMOST system detection.

In some embodiments, the reagent contains 0.75 mg/ml of acridine orange and 1.9 mg/ml of Zwittergent 3-14 for iMOST system detection.

In some embodiments, the reagent contains 0.75 mg/ml of acridine orange and 3.8 mg/ml of Zwittergent 3-14 for iMOST system detection.

In some embodiments, the reagent contains 0.05-5 mg/ml of Acridine orange range and Zwittergent 3-14.

In some embodiments, acridine orange and zwittergent 3-14 are pre-coated on a Q-card.

In some embodiments, 5 μl to 10 μl of the reagent with 0.6 mg/mL to 0.9 mg/mL of AO and 1 mg/mL to 5 mg/mL of Zwittergent is coated onto a Q-Card with a size of 10 mm to 20 mm.

In some embodiments, the cell stain agent for WBC differential comprises a fluorescence stain. Types of the fluorescent stain include but not being limited to Acridine orange dye, 3,3-dihexyloxacarbocyanine (DiOC6), Propidium Iodide (PI), Fluorescein Isothiocyanate (FITC) and Basic Orange 21 (BO21) dye, Ethidium Bromide, Brilliant Sulfaflavine and a Stilbene Disulfonic Acid derivative, Erythrosine B or trypan blue, Hoechst 33342, Trihydrochloride, Trihydrate, or DAPI (4′, 6-Diamidino-2-Phenylindole, Dihydrochloride), or any combinations thereof.

In some embodiments, the cell stain agent for WBC differential comprises Wright's stain (Eosin, methylene blue), Giemsa stain (Eosin, methyleneblue, and Azure B), May-Grünwald stain, Leishman's stain (“Polychromed” methylene blue (i.e., demethylated into various azures) and eosin), Erythrosine B stain (Erythrosin B), or any combinations thereof.

In some embodiments, the reagent comprises a cell separation agent. In some embodiments, the cell separation agent comprises, as listed in Table 1 below, for example, surfactant, Zwitterionic detergent (such as ZWITTERGENT® 3-08, ZWITTERGENT® 3-10, ZWITTERGENT® 3-12, ZWITTERGENT® 3-14, ZWITTERGENT® 3-16), CHAPS, 11b, 11c, 11d, CTAC, Tween 20, Tween 40, Tween 60, Tween 80, SLS, CTAB, or any combinations thereof.

TABLE 1
cell separation agent

| | | |
|---|---|---|
| Anionic | Alkyl Sulfates | Lithium dodecyl sulfate, Lithium dodecyl sulfate, Lithium |
| Detergent | | dodecyl sulfate, Niaproof ®, Sodium 2-ethylhexyl sulfate, |
| | | Sodium dodecyl sulfate, Sodium octyl sulfate, Teepol ™ |
| | | 610 S anionic, Turkey red oil sodium salt |
| | Alkyl | 1-Octanesulfonic acid sodium salt, 4-Dodecylbenzenesulfonic |
| | Sulfonates | acid, Ethanesulfonic acid sodium salt, Sodium 1- |
| | | butanesulfonate, Sodium 1-decanesulfonate, Sodium 1- |
| | | heptanesulfonate, Sodium 1-nonanesulfonate, Sodium 1- |
| | | octanesulfonate, Sodium 1-pentanesulfonate, Sodium 1- |
| | | propanesulfonate, Sodium hexanesulfonate, Sodium |
| | | pentanesulfonate, |
| | Bile Salts | Chenodeoxycholic acid diacetate methyl ester, |
| | | Chenodeoxycholic acid, Cholic acid, Deoxycholic acid, |
| | | Glycocholic acid hydrate, Sodium chenodeoxycholate, |
| | | Sodium cholate hydrate, Sodium cholate hydrate, Sodium |
| | | cholate hydrate, Sodium cholesteryl sulfate, Sodium |
| | | deoxycholate, Sodium glycochenodeoxycholate, Sodium |
| | | glycocholate, Sodium glycodeoxycholate, Sodium |
| | | taurochenodeoxycholate, Sodium taurocholate, Sodium |
| | | taurodeoxycholate, Sodium taurohyodeoxycholate, Sodium |
| | | taurolithocholate, Sodium tauroursodeoxycholate, |
| | | Taurocholic acid sodium salt, Taurolithocholic acid 3-sulfate |
| | | disodium salt, Ursodeoxycholic acid |
| | Other Anionic | Dicyclohexyl sulfosuccinate sodium salt, Dihexadecyl |
| | Detergents | phosphate, Dihexyl sulfosuccinate sodium salt, Docusate |
| | | sodium, Lithium 3,5-diiodosalicylate, N-Lauroylsarcosine |
| | | sodium salt, N-Lauroylsarcosine, N-Lauroylsarcosine purum, |
| | | Sodium octanoate, Triton ™ QS-15 |

| | |
|---|---|
| Cationic | Alkyltrimethylammonium bromide, Amprolium hydrochloride, Benzalkonium |
| Detergents | chloride, Benzethonium hydroxide, Benzyldimethyldodecylammonium, |
| | Benzyldimethylhexadecylammonium, Benzyldodecyldimethylammonium, |
| | Cetylpyridinium chloride, Dimethyldioctadecylammonium, |
| | Dodecylethyldimethylammonium, Dodecyltrimethylammonium, |
| | Ethylhexadecyldimethylammonium bromide, Girard's reagent T, Hexadecyl(2- |
| | hydroxyethyl)dimethylammonium dihydrogen phosphate, Hexadecylpyridinium |
| | bromide, Hexadecylpyridinium chloride, Hexadecyltrimethylammonium, |
| | Luviquat ™ FC 370, Luviquat ™ FC 550, Luviquat ™ HOLD, Luviquat ™ |
| | Mono LS, Methylbenzethonium chloride, Myristyltrimethylammonium, |
| | Tetraheptylammonium bromide, Tetrakis(decyl)ammonium bromide, Tri-C8-10- |
| | alkylmethylammonium chloride, Tridodecylmethylammonium chloride |
| | Selectophore ™ |
| Non-ionic | 1-Oleoyl-rac-glycerol, 2-Cyclohexylethyl β-D-maltoside, 4-Nonylphenyl- |
| Detergent | polyethylene glycol non-ionic, 5-Cyclohexylpentyl β-D-maltoside, 6- |
| | Cyclohexylhexyl β-D-maltoside, n-Dodecanoylsucrose, n-Dodecyl-β-D- |
| | glucopyranoside, n-Dodecyl-β-D-maltoside, n-Nonyl-β-D-glucopyranoside, n- |
| | Octyl-β-D-thioglucopyranoside, n-Decanoylsucrose, n-Decyl-β-D- |
| | maltopyranoside, n-Octanoylsucrose, n-Octyl-β-D-glucopyranoside, APO-10, |
| | APO-12, BRIJ ® O20, BRIJ ® 35, Big CHAP, Deoxy, Brij ^{®} 58, Brij ® L23, |
| | Brij ® L4, Brij ® O10, Cremophor EL ®, C₁₂E₈, C₁₂E₉, DGEA, Decaethylene |
| | glycol mono-dodecyl ether nonionic surfactant, Decyl β-D-glucopyranoside, |
| | Decyl β-D-maltopyranoside, Decyl-β-D-1-thiomaltopyranoside, Decyl-β-D- |
| | maltoside, Diethylene glycol, Digitonin, Digitoxigenin, ELUGENT ™ |
| | Detergent, Ethylene glycol, GC Stationary Phase phase Synperonic PE/F68, GC |
| | Stationary Phase phase Synperonic PE/L64, GENAPOL ® X-100, Genapol ® C- |
| | 100, Genapol ® X-080, Genapol ® X-100, Glucopone 600 CS UP, |
| | HECAMEG ®, Hexaethylene glycol monododecyl ether, Hexaethylene glycol |
| | monohexadecyl ether, Hexaethylene glycol monotetradecyl ether, Hexyl β-D- |
| | glucopyranoside, IGEPAL ® CA-630, IGEPAL ® CA-720, IPTG, Imbentin |
| | AGS/35, Isopropyl β-D-1-thiogalactopyranoside, Kolliphor ® EL, Lutrol ® OP |
| | 2000 non-ionic, Methoxypolyethylene glycol 350, Methyl 6-O-(N- |
| | heptylcarbamoyl)-α-D-glucopyranoside, N,N-Bis[3-(D- |
| | gluconamido)propyl]deoxycholamide, N,N-Dimethyldecylamine N-oxide, N,N- |
| | Dimethyldodecylamine N-oxide, N-Decanoyl-N-methylglucamine, N-Lauroyl- |
| | L-alanine, N-Nonanoyl-N-methylglucamine, N-Octanoyl-N-methylglucamine, |
| | NP-40 Alternative, Nonaethylene glycol monododecyl ether nonionic surfactant, |
| | Nonidet ™ P 40, Nonyl β-D-glucopyranoside, Nonyl β-D-maltoside, Nonyl-β-D- |
| | 1-thiomaltoside, Nonylphenyl-polyethyleneglycol acetate, Octaethylene glycol |
| | monodecyl ether, Octaethylene glycol monododecyl ether, Octaethylene glycol |
| | monohexadecyl ether, Octyl β-D-1-thioglucopyranoside, Octyl β-D- |
| | glucopyranoside, Octyl-α/β-glucoside, Octyl-β-D-glucopyranoside, |
| | PLURONIC ® F-127, Pentaethylene glycol monodecyl ether, Pluronic ® F-127, |
| | Poloxamer 188, Poloxamer 407, Poly(ethylene glycol) methyl ether, |
| | Polyoxyethylene (10) tridecyl ether mixture of C11 to C14 iso-alkyl ethers, |
| | Polyoxyethylene (20) sorbitan monolaurate, Polyoxyethylene (40) stearate, |
| | Polysorbate 20, Polysorbate 60, Polysorbate 80, SODOSIL ™ RM 003, |
| | SODOSIL ™ RM 01, diethylene glycol octadecyl ether, Saponin, Span ® 20, |
| | Span ® 60, Span ® 65, Span ® 80, Span ® 85, Sucrose monodecanoate, Sucrose |
| | monolaurate, Synperonic ® F 108, Synperonic ® PE P105, TERGITOL ™ TMN |
| | 10, TERGITOL ™ TMN 6, TERGITOL ™ solution Type NP-40, TERGITOL ™ |
| | MIN FOAM, TERGITOL ™ Type 15-S-5, TERGITOL ™ Type 15-S-7, |
| | TERGITOL ™ Type 15-S-9, TERGITOL ™ Type NP-10, TERGITOL ™ Type |
| | NP-9, TRITON ® X-100, TRITON ® X-114, TWEEN ® 20, TWEEN ® 40, |
| | TWEEN ® 60, TWEEN ® 65, TWEEN ® 80, TWEEN ®85, Tetradecyl-β-D- |
| | maltoside, Tetraethylene glycol monododecyl ether, Tetraglycol, |
| | Tetramethylammonium hydroxide pentahydrate, Thesit ®, Tridecyl β-D- |
| | maltoside, Triethylene glycol monodecyl ether, Triton ™ N-57, Triton ™ N-60, |
| | Triton ™ X-100, Triton ™ X-102, Triton ™ X-114, Triton ™ X-165, Triton ™ |
| | X-305, Triton ™ X-405, Triton ™ X-45, Tween ® 20, Tween ® 40, Tween ® 60, |
| | Tween ® 80, Tween ® 85, Tyloxapol, Undecyl β-D-maltoside, n-Dodecyl β-D- |
| | glucopyranoside, n-Dodecyl β-D-maltoside, n-Heptyl β-D-glucopyranoside, n- |
| | Heptyl β-D-thioglucopyranoside, n-Hexadecyl β-D-maltoside, n-Octyl β-D- |
| | maltoside |
| Zwitterionic | 3-(4-tert-Butyl-1-pyridinio)-1-propanesulfonate, 3-(N,N- |
| (ampholytic) | Dimethylmyristylammonio)propanesulfonate, 3-(N,N- |
| | Dimethyloctadecylammonio)propanesulfonate, 3-(N,N- |
| | Dimethyloctylammonio)propanesulfonate, 3-(N,N- |
| | Dimethylpalmitylammonio)propanesulfonate, 3-(1-Pyridinio)-1- |
| | propanesulfonate, 3-(Benzyldimethylammonio)propanesulfonate, 3- |
| | (Decyldimethylammonio)-propane-sulfonate inner salt, 3-[N,N-Dimethyl(3- |
| | palmitoylaminopropyl)ammonio]-propanesulfonate, L-α- |
| | Lysophosphatidylcholine from Glycine max (soybean), ASB-14, zwitterionic |
| | amidosulfobetaine. ASB-16 zwitterionic amidosulfobetaine detergent, ASB- |
| | C80, ASB-C8Ø, C7BzO, CHAPS, CHAPSO, DDMAB, |
| | Dimethylethylammoniumpropane, EMPIGEN ® BB Detergent, Miltefosine, N- |
| | Dodecyl-N,N-dimethyl-3-ammonio-1-propanesulfonate, N-Dodecyl-N,N- |
| | dimethyl-3-ammonio-1-propanesulfonate, O-(Decylphosphoryl)choline, |
| | Poly(maleic anhydride-alt-1-decene), 3-(dimethylamino)-1-propylamine |
| | derivative, Poly(maleic anhydride-alt-1-tetradecene), 3-(dimethylamino)-1- |
| | propylamine derivative, Sodium 2,3-dimercaptopropanesulfonate, Surfactin |
| | from Bacillus subtilis, ZWITTERGENT ® 3-08, ZWITTERGENT ® 3-10, |
| | ZWITTERGENT ® 3-12, ZWITTERGENT ® 3-14, ZWITTERGENT ® 3-16, |

### Morphology

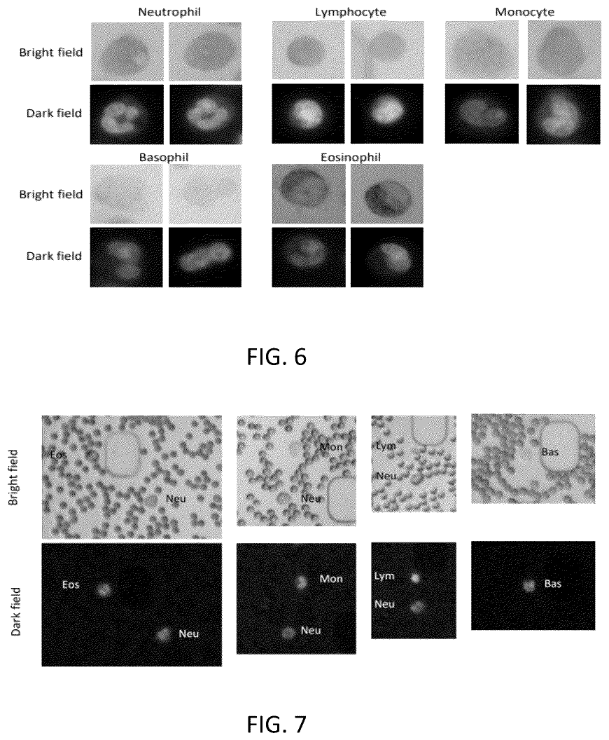

FIG. 6 shows microscopic images of white blood cells stained by acridine orange under bright and fluorescence fields, in accordance with some embodiments. The microscopic images are acquired in accordance with the following method. 5 μl of fresh whole venous blood was dropped on a plate area precoated with acridine (0.6 mg/ml) and zwittergent (1.2 mg/ml) in a Q-card and incubated at room temperature for 1 min. Bright-field and fluorescence images (Ex. 475 nm) are taken under a microscope. Cytoplasm staining and cell size are shown and analyzed using bright-field images. Fluorescent images show nuclei and cell boundaries. Detailed white blood cell features are summarized in Table 2.

FIG. 7 shows iMOST images of acridine orange-stained white blood cells under bright and fluorescence fields, in accordance with some embodiments. The iMOST images are obtained in accordance with the following images. 5 μl of fresh whole venous blood was dropped on a Q-card precoated with 0.9 mg/ml of acridine and 1 mg/ml of zwittergent and incubated at room temperature for 1 min. Bright-field and fluorescence images (Ex. blue LED at 475 nm) were taken using iMOST device. Cytoplasm staining and cell size are shown and analyzed using bright-field images, and specific acridine orange intracellular composed fluorescent color (red and green) and nuclei structures are shown and analyzed using fluorescence (fluorescent) field images. Detailed white blood cell features are summarized in Table 3.

TABLE 2
Features of acridine orange-stained WBC microscopic
features under bright/fluorescence fields

| | | | Cell size in |
|---|---|---|---|
| | | | diameter/N/C |
| | Bright Field | Fluorescence Field | ratio* (reference) |
| Lymphocytes | Bright Orange color, | Mono round or | 7-8 μm, or 12-18 μm. |
| | no to some brown-red | oval nuclei | N/C: 5:1, some 2:1 |
| | colored granules | | |
| Monocytes | Faint yellow color, cell | Mono bean/kidney | 12-20 μm |
| | edge not sharp | shaped nuclei | N/C: 3:1 to 2:1 |
| Neutrophil | Orange to bright orange color, | 2 to 5 lobed nuclei | 9-15 um |
| | faint to some brown granules | | N/C: 5:1 to 3:1 |
| Eosinophil | Orange to bright orange color, | Mostly 2 lobed | 9-15 μm |
| | with bright brown-red colored | nuclei, can be more | N/C: 1:3 (?) |
| | granules | lobes | |
| Basophil | N/A | N/A | 10-16 μm |
| | | | N/C: NA |

TABLE 3
Features of acridine orange-stained WBC U2D-Mi9
under bright/fluorescent(fluorescence) fields

| | | Fluorescent | Cell size in diameter/ |
|---|---|---|---|
| | Bright Field | (Fluorescence) Field | N/C ratio* (reference) |
| Lymphocytes | Faint yellow stained color, | Mono round or oval | 7-8 μm, or 12-18 μm. |
| | cell small and round or oval | nuclei, cell in green | N/C: 5:1, some 2:1 |
| | shaped | color | |
| Monocytes | Faint yellow stained color, | Mono bean/kidney | 12-20 μm |
| | cell edge not sharp, cell shape | shaped nuclei, cell in | N/C: 3:1 to 2:1 |
| | round, oval or irregular. | yellow color | |
| Neutrophil | Orange to bright orange | 2 to 5 lobed nuclei, | 9-15 μm |
| | stained color, faint to some | cell in red color | N/C: 5:1 to 3:1 |
| | brown granules, cell shape | | |
| | round or oval. | | |
| Eosinophil | Orange to bright orange | Mostly 2 lobed | 9-15 μm |
| | color, with bright brown-red | nuclei, can be more | N/C: 1:3 (?) |
| | colored granules, cell shape | lobes, cell in red | |
| | round or oval. | color | |
| Basophil | Faint or no staining, cell | Mostly 2 lobed | 10-16 μm |
| | shape not round or oval. | nuclei, cell in green | N/C: NA |
| | | color (mainly nuclei | |
| | | color) | |

Besides the features listed above, the features for distinguishing white blood cell types include but are not limited to the number of lobes, cell area, cell shape, nucleus area, ratio of nucleus, nucleus shape, and optical absorption (intensity) and texture of cell and components of cells at a given wavelength.

### Cell Classification and Machine Learning

With respect to cell detection and differentiation, image patches containing cells are first located and extracted from the image of a sample, in accordance with some embodiments. The cell patches are labeled by their type and saved for training a deep learning model to classify cells into different types. At an inference stage, extracted cell patches from the image of the sample are fed into the trained deep learning model for cell-type classification.

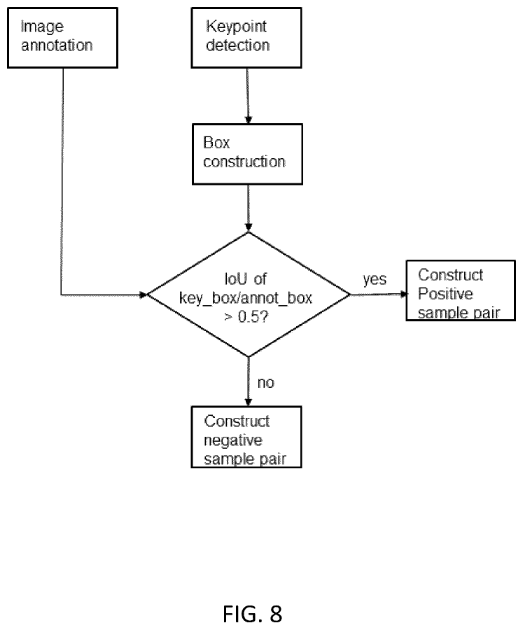

In some embodiments, paired bright-field (BF) and fluorescence-field (FF) images are captured. The FF images can be used for cell detection and localization. In some embodiments, images are first labeled by an expert using image a label tool such as, for example, LabelImage. In some embodiments, for each cell, a bounding box is drawn, and the type of the cell is identified. In some embodiments, the size and location of the bounding box and the cell type are recorded in a file. In some embodiments, the file is a .xml file. In some embodiments, each pair of BF and FF images has a corresponding file, for example, .xml file, to identify the locations and types of the cells on the image pair. In some embodiments, cell patches can be extracted based on the .xml files. In some embodiments, the key points corresponding to the cells in the sample are detected using methods such as blob detection, and bounding boxes are then constructed using the key points as centers. In some embodiments, the bounding boxes can have the same size or different sizes based on the blob size that captures the detected cells. Further refinement of the detection can be performed using the intersection over union (IoU) test with all the boxes detected for containing cells. In some embodiments, the refinement can be performed using the IoU test with all the boxes determined from blob detection around key points for containing cells and the boxes recorded in the corresponding .xml file. As illustrated in FIG. 8, if the IoU is above a certain threshold, the image patch cropped using the box constructed from the key point is assumed as a positive sample of the cell. In some embodiments, the cell is labeled as the type of the corresponding box in the .xml file that passed the IoU test. Otherwise, the image patch is regarded as a negative sample.

At each key point, paired FF and BF cell image patches can be extracted from the paired FF and BF images of the sample, respectively. As such, the extracted paired FF and BF cell image patches form a matching image pair (BF_cell, FF_cell) for cell classification.

In some embodiments, FF and BF cell image patch pairs are first extracted based on key point detection, these cell image patch pairs are then labeled by an expert into different cell types.

The set of labeled FF and BF cell image patch pairs are split into a training set and a test set for training and testing a deep learning machine model, respectively.

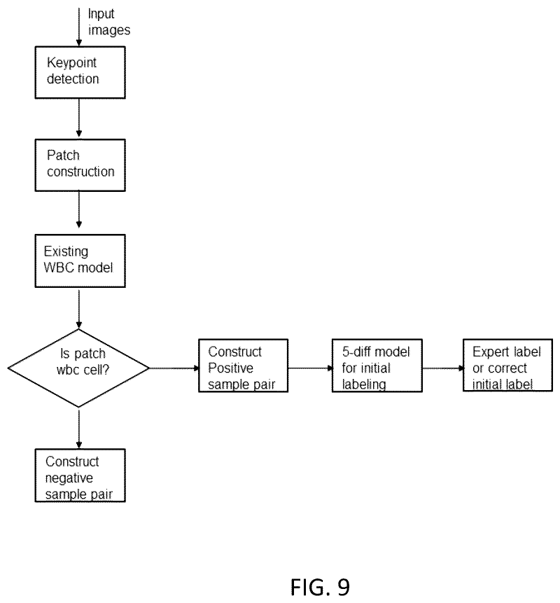

In some embodiments, cell image patches are extracted using a machine learning model as shown in FIG. 9. FF image patches around key points in the image of the sample are collected. The FF cell patches are first fed to a second WBC classification model to detect if a white blood cell exists in the image patch. If the cell patch is classified as containing a WBC cell, a paired BF cell image patch is then constructed from the BF image at the same location. As such, the FF and BF image patches of the cell are therefore aligned. The paired FF and BF cell image patches are then used for cell-type classification in WBC differentials.

In some embodiments, the paired FF and BF image patch of the cell is fed into a 5-diff model for initial labeling by machine. However, as an initial model is trained with only limited data, the labeling from the initial machine learning model is verified by area experts, and the paired image of the cell with the verified annotation is then added to the training set to refine the machine learning model or added to the validation data set for model testing.

In some embodiments, after cell segmentations, some predetermined features are extracted from each cell, including but not being limited to the number of lobes, cell area, ratio of nucleus, nucleus shape, and optical absorption of cell at a given wavelength. Such extracted features of training samples are used to train a classifier model, which is then applied to the extracted features of test samples to classify the WBC types.

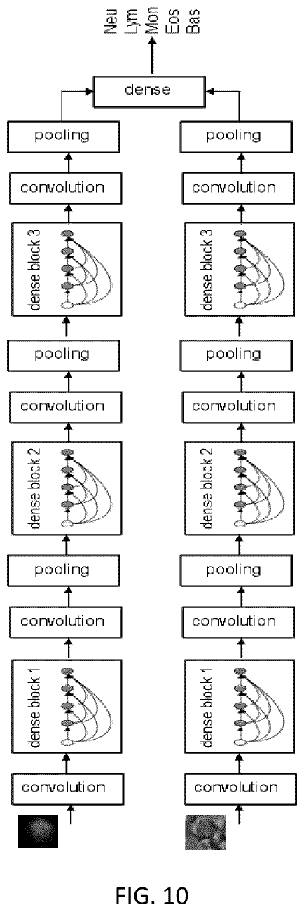

FIG. 10 shows a machine learning model architecture in accordance with some embodiments. The machine learning model is built based on the network structure of DenseNet with input features extracted from both FF and BF cell image patches. In some embodiments, the extracted features from the paired cell image patches can be combined at the fully connected layer for cell-type classification.

In some embodiments, a 5-way classification model can be obtained, which classifies the white blood cells in the whole blood sample into NEU, LYM, MON, EOS, and BAS classes. In some embodiments, a 6-way classification model can be obtained, including a negative non-white blood cell class in addition to NEU, LYM, MON, EOS, and BAS classes.

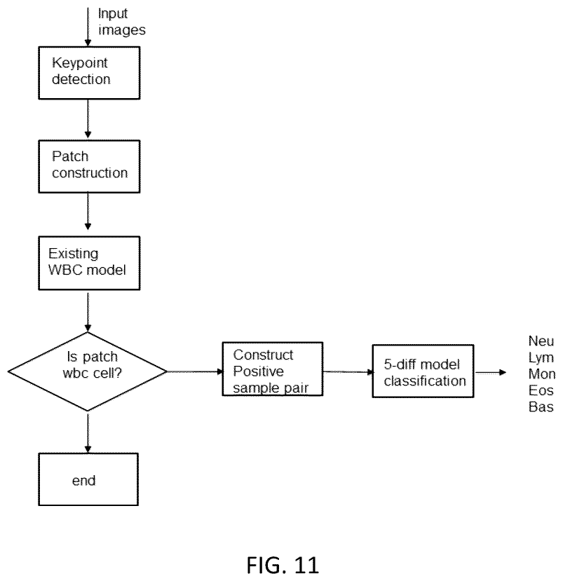

For cell-type inference, if a 5-class classification model is used, WBC detection model can be used first to detect and locate WBC in the FF image of the sample in accordance with some embodiments. The paired cell patches are then extracted from the FF and BF images respectively to form the matching pair image of the cell, which is then used as the input to 5-class classification model to determine the cell types as shown in FIG. 11.

If a 6-class classification model is used, key point detection can be used to determine the matched image patch pairs of the cell, in accordance with some embodiments. The matched image pairs of the cell are fed to the 6-class classification model, which classifies the cells into NEU, LYM, MON, EOS, BAS, and a negative (background) class for not containing a white blood cell.

In some embodiments, the matched image pair of the cell are grouped together as an extended vector, (BF_cell, FF_cell), for the cell classification/differentiation machine learning model to determine the cell type in the paired image patches.

In some embodiments, the images of the whole blood sample in the sample holder are taken by an imager with a lower resolution than the high-resolution microscope. Therefore, additional steps of image normalization topology transform are performed to correct distortions from the lower quality imager based on the known shape/location of the monitoring marks, such as pillars, fabricated on the sample holder. As such, the cell detection and classification are performed in the normalized sample space with less distortion.

In some embodiments, it uses super resolution transform which maps the image of the sample taken by a lower resolution imager to the higher resolution microscope domain for cell classification and differentiation.

In some embodiments, the matching BF_cell and FF_cell images are merged by adding together into one vector to reduce the input dimension for cell classification and differentiation.

In some embodiments, supervised machine learning is applied and extended to build the training database, wherein a small number of cells are first labeled by an expert in the field and these supervise labeled data are used as the seed data to generate the first machine learning model for cell classification and differentiation. The first machine learning model is used to classify more unlabeled data, wherein the labeling from the machine learning model is then verified by the area experts to generate more labeled data to train and build a higher-quality model. This process can repeat until a sufficient amount of labeled data is obtained for the machine learning model training purpose.

To reduce the adverse effects of using lower resolution consumer camera, such as the camera in the smartphone, in cell detection and classification such as the WBC differentiation, some embodiments use the method including the steps of: receiving a first image, captured by a first optical sensor, of a sample holder containing a sample, wherein the sample holder is fabricated with a patterned structural elements at predetermined positions; identifying a first region in the first image based on locations of one or more structural elements of the patterned structural elements in the first image; determining a spatial transform associated with the first region based on a mapping between the locations of the one or more structural elements in the first image and predetermined positions of one or more structural elements in the sample holder; applying the spatial transform to the first region in the first image to calculate a transformed first region; and training the machine learning model using the transformed first image.

In some embodiments, the device or sample holder comprises a first plate, a second plate, and the patterned structural element. The patterned structural element comprises pillars embedded at predetermined or known positions on at least one of the first and second plates.

In some embodiments, the method further includes the steps of: detecting the locations of the patterned structural elements in the first image; partitioning the first image into regions comprising the first region, wherein each of the regions is defined by four structural elements at four corners of the corresponding region; determining a corresponding spatial transform associated with each of the regions in the first image based on a mapping between the locations of the four structural elements at the four corners of the corresponding region and the four predetermined positions of the four structural elements in the sample holder; applying the corresponding spatial transform to each of the regions in the first image to calculate a corresponding transformed region in the first image; and training the machine learning model using each of transformed regions in the first image, wherein the trained machine learning model is used to detect the cells from the image of the sample and differentiate them into different cell classes based on the transformed image.

In some embodiments, the predetermined positions of the patterned structural elements are distributed periodically with at least a periodicity value, and wherein detecting the locations of the patterned structural elements in the first image includes: detecting, using a second machine learning model, the locations of the patterned structural elements in the first image; and correcting, based on the at least one periodicity value, an error in the detected locations of the patterned structural elements in the first image.

In some embodiments, it provides a method for converting an assay image using a machine learning model, the method including the steps of: receiving a first image, captured by a first optical sensor, of a sample holder containing a sample, wherein the sample holder is fabricated with a patterned structural elements at predetermined positions; identifying a first region in the first image based on locations of one or more structural elements of the patterned structural elements in the first image; determining a spatial transform associated with the first region based on a mapping between the locations of the one or more structural elements in the first image and predetermined positions of one or more structural elements in the sample holder; applying the spatial transform to the first region in the first image to calculate a transformed first region; training a machine learning model based on the transformed first region, and applying the machine learning model to detect and differentiate cells from the transformed region of the image of the sample.

In some embodiments, the method further comprises: partitioning the first image into a plurality of regions based on the locations of the one or more structural elements of the patterned structural elements in the first image, wherein the plurality of regions comprises the first region; determining a respective spatial transform associated with each of the plurality of regions; applying the corresponding spatial transform to each of the plurality of regions in the first image to calculate transformed regions; applying the machine learning model to each of the transformed regions in the first image to perform cell detection and differentiation.

In some embodiments, the disclosure provides an image-based assay system comprising: a database system to store images; and a processing device, communicatively coupled to the database system, to: receive a first image, captured by a first optical sensor, of a sample holder containing a sample, wherein the sample holder is fabricated with patterned structural elements at predetermined positions; identify a first region in the first image based on locations of one or more structural elements of the patterned structural elements in the first image; determine a spatial transform associated with the first region based on a mapping between the locations of the one or more structural elements in the first image and predetermined positions of one or more structural elements in the sample holder; apply the spatial transform to the first region in the first image to calculate a transformed first region; and train the machine learning model using the transformed first image.

In some embodiments, the sample holder comprises a first plate, a second plate, and the patterned structural elements, and wherein the patterned structural elements comprise pillars embedded at the predetermined positions on at least one of the first plate or the second plate.

In some embodiments, the processing device is further to: detect the locations of the patterned structural elements in the first image; partition the first image into regions comprising the first region, wherein each of the regions is defined by four structural elements at four corners of the corresponding region; determine a corresponding spatial transform associated with each of the regions in the first image based on a mapping between the locations of the four structural elements at the four corners of the corresponding region and the four predetermined positions of the four structural elements in the sample holder; apply the corresponding spatial transform to each of the regions in the first image to calculate a corresponding transformed region in the first image; and train the machine learning model using each of transformed regions in the first image, wherein the trained machine learning model is used to do cell detection and differentiation.

In some embodiments, the predetermined positions of the patterned structural elements are distributed periodically with at least one periodicity value, and wherein to detect the locations of the patterned structural elements in the first image, the processing device is further to: detect, using a second machine learning model, the locations of the patterned structural elements in the first image; and correct, based on the at least one periodicity value, an error in the detected locations of the patterned structural elements in the first image.

It is appreciated that the device, system, and method in this disclosure may apply to the identification of other cells than white blood cells with or without apparent modification. Such modification should be understood as being within the scope of this disclosure.

With regard to the preceding description, it is to be understood that changes may be made in detail, especially in matters of the construction materials employed and the shape, size, and arrangement of parts without departing from the scope of the present disclosure. This specification and the embodiments described are exemplary only, with the true scope and spirit of the disclosure being indicated by the claims that follow.

### ASPECTS

Any one of aspects 1-35 is combinable with any one of aspects 36-73, and any one of aspects 36-71 is combinable with any one of aspects 72-73
Aspect 1. A device for identifying a bio-entity including a cell type and count in a sample, comprising a first plate, a second plate, and a patterned structural element,
   wherein the first and second plates are movable relative to each other and form different configurations, including an open configuration and a closed configuration,
   the open configuration is a configuration in which the first and second plates are at least partially separate from each other, and
   the closed configuration is a configuration in which the first and second plate are compressed on each other;
Aspect 2. The device of Aspect 1, wherein the patterned structural element comprises a plurality of spacers affixed at least one of the first and second plates, and the spacers that are distributed periodically with at least one periodicity value.
Aspect 3. The device of Aspect 2, wherein the spacers have a height of 1 μm to 150 μm.
Aspect 4. The device of any of Aspects 2-3, wherein the spacers have a height of 2 μm to 30 μm.
Aspect 5. The device of any of Aspects 2-4, wherein the spacers have an inter-spacer distance of 10 μm to 1000 μm.
Aspect 6. The device of any of Aspects 2-5, wherein the spacers have an inter-spacer distance of 50 μm to 200 μm.
Aspect 7. The device of any of Aspects 2-6, wherein the spacers have a rectangle shape with round corners.
Aspect 8. The device of any of Aspects 2-7, wherein the spacers are disposed on the first plate.
Aspect 9. The device of any of Aspects 1-8, wherein the first and second plates comprise poly(methyl methacrylate).
Aspect 10. The device of any of Aspects 1-9, wherein at least one of the first and second plates comprises a sample deposition site.
Aspect 11. The device of any of Aspects 1-10, wherein the first plate comprises a sample deposition site.
Aspect 12. The device of any of Aspects 1-11, wherein the second plate comprises a sample deposition site.
Aspect 13. The device of any of Aspects 1-12, wherein the sample deposition site is coated with a reagent.
Aspect 14. The device of any of Aspect 13, wherein the reagent comprises a staining reagent and/or a detergent.
Aspect 15. The device of Aspect 13, wherein the reagent comprises a staining reagent.
Aspect 16. The device of any of Aspect 13-15, wherein the reagent comprises a staining reagent and a detergent.
Aspect 17. The device of any of Aspects 14-16, wherein the detergent is capable of dispersing cells.
Aspect 18. The device of Aspect 14, wherein the staining reagent comprises a fluorescent stain.
Aspect 19. The device of Aspects 18, wherein the fluorescent stain a fluorescence stain comprises at least one selected from the group consisting of acridine orange dye, 3,3-dihexyloxacarbocyanine (DiOC6), Propidium Iodide (PI), Fluorescein Isothiocyanate (FITC) and Basic Orange 21 (BO21) dye, Ethidium Bromide, Brilliant Sulfaflavine and a Stilbene Disulfonic Acid derivative, Erythrosine B or trypan blue, Hoechst 33342, Trihydrochloride, Trihydrate, and DAPI (4′, 6-Diamidino-2-Phenylindole, Dihydrochloride).
Aspect 20. The device of Aspect 14, wherein the staining agent comprises at least one selected from the group consisting of a Wright's stain, a Giemsa stain, a May-Grunwald stain, a Leishman's stain, and Erythrosine B stain.
Aspect 21. The device of Aspect 20, wherein Wright's stain comprises at least one selected from the group consisting of Eosin and methylene blue, and
   the Giemsa stain comprises at least one selected from the group consisting of Eosin, methylene blue, and Azure B.
Aspect 22. The device of Aspect 14, wherein the detergent comprises at least one selected from a Zwitterionic detergent, an anionic detergent, a cationic Detergent, and a non-ionic detergent.
Aspect 23. The device of Aspect 22, wherein the Zwitterionic detergent comprises at least one selected from the group consisting of 3-(4-tert-Butyl-1-pyridinio)-1-propanesulfonate, 3-(N,N-Dimethylmyristylammonio)propanesulfonate, 3-(N,N-Dimethyloctadecylammonio)propanesulfonate, 3-(N,N-Dimethyloctylammonio)propanesulfonate, 3-(N,N-Dimethylpalmitylammonio)propanesulfonate, 3-(1-Pyridinio)-1-propanesulfonate, 3-(Benzyldimethylammonio)propanesulfonate, 3-(Decyldimethylammonio)-propane-sulfonate inner salt, 3-[N,N-Dimethyl(3-palmitoylaminopropyl)ammonio]-propanesulfonate, L-α-Lysophosphatidylcholine from Glycine max (soybean), ASB-14, zwitterionic amidosulfobetaine. ASB-16 zwitterionic amidosulfobetaine detergent, ASB-C80, ASB-C8Ø, C7BzO, CHAPS, CHAPSO, DDMAB, Dimethylethylammoniumpropane, EMPIGEN® BB Detergent, Miltefosine, N-Dodecyl-N,N-dimethyl-3-ammonio-1-propanesulfonate, N-Dodecyl-N,N-dimethyl-3-ammonio-1-propanesulfonate, O-(Decylphosphoryl)choline, Poly(maleic anhydride-alt-1-decene), 3-(dimethylamino)-1-propylamine derivative, Poly(maleic anhydride-alt-1-tetradecene), 3-(dimethylamino)-1-propylamine derivative, Sodium 2,3-dimercaptopropanesulfonate, Surfactin from Bacillus subtilis, ZWITTERGENT® 3-08, ZWITTERGENT® 3-10, ZWITTERGENT® 3-12, ZWITTERGENT® 3-14, and ZWITTERGENT® 3-16.
Aspect 24. The device of Aspect 22, wherein the non-ionic detergent comprises at least one selected from the group consisting of 1-Oleoyl-rac-glycerol, 2-Cyclohexylethyl β-D-maltoside, 4-Nonylphenyl-polyethylene glycol non-ionic, 5-Cyclohexylpentyl β-D-maltoside, 6-Cyclohexylhexyl β-D-maltoside, n-Dodecanoylsucrose, n-Dodecyl-β-D-glucopyranoside, n-Dodecyl-β-D-maltoside, n-Nonyl-β-D-glucopyranoside, n-Octyl-β-D-thioglucopyranoside, n-Decanoylsucrose, n-Decyl-β-D-maltopyranoside, n-Octanoylsucrose, n-Octyl-β-D-glucopyranoside, APO-10, APO-12, BRIJ® O20, BRIJ® 35, Big CHAP, Deoxy, Brij® 58, Brij® L23, Brij® L4, Brij® 010, Cremophor EL®, C₁₂E₈, C₁₂E₉, DGEA, Decaethylene glycol mono-dodecyl ether nonionic surfactant, Decyl β-D-glucopyranoside, Decyl β-D-maltopyranoside, Decyl-3-D-1-thiomaltopyranoside, Decyl-3-D-maltoside, Diethylene glycol, Digitonin, Digitoxigenin, ELUGENT™ Detergent, Ethylene glycol, GC Stationary Phase phase Synperonic PE/F68, GC Stationary Phase phase Synperonic PE/L64, GENAPOL® X-100, Genapol® C-100, Genapol® X-080, Genapol® X-100, Glucopone 600 CS UP, HECAMEG®, Hexaethylene glycol monododecyl ether, Hexaethylene glycol monohexadecyl ether, Hexaethylene glycol monotetradecyl ether, Hexyl 3-D-glucopyranoside, IGEPAL® CA-630, IGEPAL® CA-720, IPTG, Imbentin AGS/35, Isopropyl β-D-1-thiogalactopyranoside, Kolliphor® EL, Lutrol® OP 2000 non-ionic, Methoxypolyethylene glycol 350, Methyl 6-O—(N-heptylcarbamoyl)-α-D-glucopyranoside, N,N-Bis[3-(D-gluconamido)propyl]deoxycholamide, N,N-Dimethyldecylamine N-oxide, N,N-Dimethyldodecylamine N-oxide, N-Decanoyl-N-methylglucamine, N-Lauroyl-L-alanine, N-Nonanoyl-N-methylglucamine, N-Octanoyl-N-methylglucamine, NP-40 Alternative, Nonaethylene glycol monododecyl ether nonionic surfactant, Nonidet™ P 40, Nonyl β-D-glucopyranoside, Nonyl 3-D-maltoside, Nonyl-3-D-1-thiomaltoside, Nonylphenyl-polyethyleneglycol acetate, Octaethylene glycol monodecyl ether, Octaethylene glycol monododecyl ether, Octaethylene glycol monohexadecyl ether, Octyl β-D-1-thioglucopyranoside, Octyl β-D-glucopyranoside, Octyl-α/β-glucoside, Octyl-β-D-glucopyranoside, PLURONIC® F-127, Pentaethylene glycol monodecyl ether, Pluronic® F-127, Poloxamer 188, Poloxamer 407, Poly(ethylene glycol) methyl ether, Polyoxyethylene (10) tridecyl ether mixture of C11 to C14 iso-alkyl ethers, Polyoxyethylene (20) sorbitan monolaurate, Polyoxyethylene (40) stearate, Polysorbate 20, Polysorbate 60, Polysorbate 80, SODOSIL™ RM 003, SODOSIL™ RM 01, diethylene glycol octadecyl ether, Saponin, Span® 20, Span® 60, Span® 65, Span® 80, Span® 85, Sucrose monodecanoate, Sucrose monolaurate, Synperonic® F 108, Synperonic® PE P105, TERGITOL™ TMN 10, TERGITOL™ TMN 6, TERGITOL™ solution Type NP-40, TERGITOL™ MIN FOAM, TERGITOL™ Type 15-S-5, TERGITOL™ Type 15-S-7, TERGITOL™ Type 15-S-9, TERGITOL™ Type NP-10, TERGITOL™ Type NP-9, TRITON® X-100, TRITON® X-114, TWEEN® 20, TWEEN® 40, TWEEN® 60, TWEEN® 65, TWEEN® 80, TWEEN®85, Tetradecyl-β-D-maltoside, Tetraethylene glycol monododecyl ether, Tetraglycol, Tetramethylammonium hydroxide pentahydrate, Thesit®, Tridecyl β-D-maltoside, Triethylene glycol monodecyl ether, Triton™ N-57, Triton™ N-60, Triton™ X-100, Triton™ X-102, Triton™ X-114, Triton™ X-165, Triton™ X-305, Triton™ X-405, Triton™ X-45, Tween® 20, Tween® 40, Tween® 60, Tween® 80, Tween® 85, Tyloxapol, Undecyl β-D-maltoside, n-Dodecyl β-D-glucopyranoside, n-Dodecyl β-D-maltoside, n-Heptyl 3-D-glucopyranoside, n-Heptyl 3-D-thioglucopyranoside, n-Hexadecyl β-D-maltoside, and n-Octyl 3-D-maltoside.
Aspect 25. The device of Aspect 22, wherein the cationic detergent comprises at least one selected from the group consisting of Alkyltrimethylammonium bromide, Amprolium hydrochloride, Benzalkonium chloride, Benzethonium hydroxide, Benzyldimethyldodecylammonium, Benzyldimethylhexadecylammonium, Benzyldodecyldimethylammonium, Cetylpyridinium chloride, Dimethyldioctadecylammonium, Dodecylethyldimethylammonium, Dodecyltrimethylammonium, Ethylhexadecyldimethylammonium bromide, Girard's reagent T, Hexadecyl(2-hydroxyethyl)dimethylammonium dihydrogen phosphate, Hexadecylpyridinium bromide, Hexadecylpyridinium chloride, Hexadecyltrimethylammonium, Luviquat™ FC 370, Luviquat™ FC 550, Luviquat™ HOLD, Luviquat™ Mono LS, Methylbenzethonium chloride, Myristyltrimethylammonium, Tetraheptylammonium bromide, Tetrakis(decyl)ammonium bromide, Tri-C8-10-alkylmethylammonium chloride, Tridodecylmethylammonium chloride Selectophore™
Aspect 26. The device of Aspect 22, wherein the anionic detergent comprises at least one selected from the group consisting of Alkyl Sulfates, Alkyl Sulfonates, and Bile Salts.
Aspect 27. The device of Aspect 13, wherein the reagent comprises acridine orange (AO) and zwittergent 3-14.
Aspect 28. The device of Aspect 27, wherein the reagent comprises 0.6 mg/ml of and 1.2 mg/ml of Zwittergent 3-14 for microscopic detection.
Aspect 29. The device of Aspect 27, wherein the reagent comprises 0.9 mg/ml of acridine orange and 1 mg/ml of Zwittergent 3-14.
Aspect 30. The device of Aspect 27, wherein the reagent comprises 0.6 mg/ml of acridine orange and 1.9 mg/ml of Zwittergent 3-14.
Aspect 31. The device of Aspect 27, wherein the reagent comprises 0.6 mg/ml of acridine orange and 2 mg/ml of Zwittergent 3-14.
Aspect 32. The device of Aspect 27, wherein the reagent comprises 0.75 mg/ml of acridine orange and 1.9 mg/ml of Zwittergent 3-14.
Aspect 33. The device of Aspect 27, wherein the reagent comprises 0.75 mg/ml of acridine orange and 3.8 mg/ml of Zwittergent 3-14.
Aspect 34. The device of Aspect 27, wherein 5 μl to 10 μl of the reagent with 0.6 mg/mL to 0.9 mg/mL of AO and 1 mg/mL to 5 mg/mL of Zwittergent is coated onto a Q-Card with a size of 10 mm to 20 mm.
Aspect 35. The device of any of Aspects 1-34, wherein the device is a Q-Card.
Aspect 36. A method for identifying a bio-entity including a cell type and count in a sample, comprising:
   (a) providing a device of any of Aspects 1-35;
   (b) depositing the sample between the first and second plates;
   (c) reducing the spacing of the first and second plates so that the first and second plates are in the closed configuration to compress the sample into a layer; and
   (d) imaging the sample to obtain an image; and
   (e) measuring and analyzing the image against a database generated with a machine learning model to obtain the bio-entity of the sample.
Aspect 37. The method of Aspect 36, wherein step (e) measures cell count and cell differential.
Aspect 38. The method of any of Aspects 36-37, wherein the sample is a blood sample containing white blood cells.
Aspect 39. The method of any of Aspects 36-38, wherein the sample is a whole blood.
Aspect 40. The method of any of Aspects 36-39, wherein the sample is a whole blood without dilution.
Aspect 41. The method of any of Aspects 36-40, wherein the sample containing WBC is capillary or venous whole blood with or without anti-agglutination as EDTA.
Aspect 42. The method of any of Aspects 36-41, wherein step (e) identifies types and amounts of white blood cells, wherein the types comprise one or more of neutrophils, lymphocytes, monocytes, eosinophils, and basophils.
Aspect 43. The method of any of Aspects 36-42, wherein the cell is pressed and deformed in the device.
Aspect 44. The method of any of Aspects 36-43, wherein the cell comprises white blood cells, and the white blood cells are pressed to a layer having a thickness of 1 μm, 2 μm, 3 μm, 5 μm, 10 μm, 15 μm or in a range between any two of the thicknesses.
Aspect 45. The method of Aspect 44, wherein the white blood cells are pressed to a layer having a thickness of 2 μm, 3 μm, 5 μm, 10 μm, or in a range between any two of the thicknesses.
Aspect 46. The method of any of Aspects 36-45, wherein the cell or other bio-entity is deformed by 1%, 5%, 10%, 20%, 30%, 40%, 50%, 60%, 70%, 80%, 90%, or a value between any two of the values.
Aspect 47. The method of Aspects 36-46, wherein the cell comprises white blood cells, and the white blood cells are pressed to achieve an area increase of 10%, 20%, 30%, 50%, 80%, 100%, 200%, 300%, 500%, 1000% or in a range between any two of the values.
Aspects 48. The method of Aspect 47, wherein the white blood cells are pressed to achieve an area increase of 50%, 80%, 100%, 200%, 300%, or in a range between any two of the values.
Aspect 49. The method of any of Aspects 36-48, wherein step (e) is performed with a machine learning model.
Aspect 50. The method of any of Aspects 36-49, wherein step (e) further comprises:
   extracting cell patches from the image of the sample, and
   feeding the cell patches into a trained deep learning model for cell-type classification,
   wherein the image comprises a paired bright-field (BF) image and fluorescence-field (FF) image of the sample.
Aspect 51. The method of Aspect 50, the step of extracting the cell patches further comprising detecting and localizing cells shown on the FF image,
   wherein key points corresponding to the cells in the sample are detected using a blob detection.
Aspect 52. The method of any of Aspects 50-51, further comprising constructing bounding boxes using the key points as centers,
   wherein the bounding boxes have the same size or different sizes based on the blob size that captures the cells.
Aspect 53. The method of any of Aspects 50-52, further comprising refining the detection and localization of the cells using an intersection over union (IoU) test with all the bounding boxes identified for containing cells,
   if the IoU is above a threshold, an image patch is cropped using the box constructed from the key point is assumed as a positive sample of the cell, otherwise the image patch is regarded as a negative sample.
   wherein, at each key point, paired BF image and FF image patches are extracted from the paired FF and BF images of the sample, respectively, and
   the extracted paired BF image and FF image patches form a matched image pair (BF_cell, FF_cell) for cell classification.
Aspect 54. The method of any of Aspects 50-53, wherein the step of extracting the cell patches comprising:
   collecting a FF image patch around a key point in the image,
   feeding the FF image patch to a second white blood cell classification model to detect if a white blood cell exists in the FF image patch,
   wherein, if the FF image patch is classified as containing a white blood cell, a paired BF image patch is constructed from the BF image at the same location so that the FF and BF image patches of the cell are aligned.
Aspect 55. The method of any of Aspects 50-54, further comprising feeding the paired FF image and BF image patch of the cell into a 5-diff model for initial labeling by machine.
Aspect 56. The method of any of Aspects 36-54, wherein the machine learning model is built based on the network structure of DenseNet with input features extracted from cell patches of a paired BF image and FF image of the sample.
Aspect 57. The method of any of Aspects 36-56, further comprising obtaining a matched image pair of a cell from the extracted paired BF image and FF image patches.
Aspect 58. The method of Aspect 57, further comprising grouping a matched image pair of the cell as an extended vector, (BF_cell, FF_cell), for the cell classification/differentiation machine learning model to determine the cell type in the paired image patches.
Aspect 59. The method of Aspect 58, the image the matched BF_cell and FF_cell images are merged by adding together into one vector to reduce the input dimension for cell classification and differentiation.
Aspect 60. The method of any of Aspects 49-59, wherein a process for building the machine learning model comprises:
   labeling a small number of cells to obtain a first labeled data that is used as a first seed data to generate a first machine learning model for cell classification and differentiation,
   classifying unlabeled data with the first machine learning model to obtain a second labeled data, and
   verifying the second labeled data to obtain a second seed data to train and build a higher quality machine learning model.
Aspect 61. The method of any of Aspects 36-60, wherein the image is captured by a mobile phone or a consumer camera, and the device is fabricated with the patterned structural element at a predetermined or known position of the device.
Aspect 62. The method of any of Aspects 36-61, wherein the method further comprises:
   receiving a first image, captured by a first optical sensor, of the device containing the sample;
   identifying a first region in the first image based on locations of one or more structural elements of the patterned structural elements in the first image;
   determining a spatial transform associated with the first region based on a mapping between the locations of the one or more structural elements in the first image and predetermined positions of one or more structural elements in the sample holder;
   applying the spatial transform to the first region in the first image to calculate a transformed first region; and
   training the machine learning model using the transformed first image.
Aspect 63. The method of any of Aspects 1-61, wherein the patterned structural element comprises pillars embedded at the predetermined positions on at least one of the first plate and the second plate.
Aspect 64. The method of any of Aspects 36-63, further comprising:
   detecting locations of a patterned structural element of the device in a first image;
   partitioning the first image into regions comprising a first region, wherein each of the regions is defined by four structural elements at four corners of the corresponding region;
   determining a corresponding spatial transform associated with each of the regions in the first image based on a mapping between the locations of the four structural elements at the four corners of the corresponding region and the four predetermined positions of the four structural elements in the sample holder;
   applying the corresponding spatial transform to each of the regions in the first image to calculate a corresponding transformed region in the first image; and
   training the machine learning model using each of the transformed regions in the first image to obtain a trained machine learning model,
   wherein the trained machine learning model is used to detect the cells from the image of the sample and differentiate them into different cell classes based on the transformed image.
Aspect 65. The method of any of Aspects 1-64, wherein the patterned structural element comprises spacers that are distributed periodically with at least one periodicity value, and
   detecting the locations of the patterned structural element in the first image comprises:
   detecting, using a second machine learning model, the locations of the patterned structural elements in the first image; and
   correcting, based on the at least one periodicity value, an error in the detected locations of the patterned structural elements in the first image.
Aspect 66. The method of any of Aspects 36-65, further comprising:
   receiving a first image, captured by a first optical sensor, of the device containing the sample;
   identifying a first region in the first image based on locations of one or more structural elements of the patterned structural elements in the first image;
   determining a spatial transform associated with the first region based on a mapping between the locations of the one or more structural elements in the first image and predetermined positions of one or more structural elements in device;
   applying the spatial transform to the first region in the first image to calculate a transformed first region;
   training the machine learning model based on the transformed first region, and applying the machine learning model to detect and differentiate cells from the transformed region of the image of the sample.
Aspect 67. The method of any of Aspects 36-66, further comprising:
   partitioning the first image into a plurality of regions based on the locations of the one or more structural elements of the patterned structural elements in the first image, wherein the plurality of regions comprises the first region;
   determining a respective spatial transform associated with each of the plurality of regions;
   applying the corresponding spatial transform to each of the plurality of regions in the first image to calculate transformed regions; and
   applying the machine learning model to each of the transformed regions in the first image to perform cell detection and differentiation.
Aspect 68. The method of any of Aspects 36-67, wherein step (e) is performed with an image-based assay system comprising:
   a database system to store images; and
   a processing device, communicatively coupled to the database system, to:
      receive a first image of the sample in the device, wherein the patterned structural element comprising one or more structural elements having predetermined positions;
      identify a first region in the first image based on locations of the one or more structural elements in the first image;
      determine a spatial transform associated with the first region based on a mapping between the locations of the one or more structural elements in the first image and predetermined positions of the one or more structural elements in device;
      apply the spatial transform to the first region in the first image to calculate a transformed first region; and
      train a machine learning model using the transformed first image.
Aspect 69. The method of Aspect 68, wherein the processing device is further to:
   detect the locations of the patterned structural element in the first image;
   partition the first image into regions comprising the first region, wherein each of the regions is defined by four structural elements at four corners of the corresponding region;
   determine a corresponding spatial transform associated with each of the regions in the first image based on a mapping between the locations of the four structural elements at the four corners of the corresponding region and the four predetermined positions of the four structural elements in the device;
   apply the corresponding spatial transform to each of the regions in the first image to calculate a corresponding transformed region in the first image; and
   train the machine learning model using each of transformed regions in the first image, wherein the trained machine learning model is used to do cell detection and differentiation.
Aspect 70. The method of any of Aspects 36-69, wherein the patterned structural element is distributed periodically with at least one periodicity value, and
   wherein to detect the locations of the patterned structural elements in the first image, the processing device is further to:
   detect, using a second machine learning model, the locations of the patterned structural elements in the first image; and correct, based on the at least one periodicity value, an error in the detected locations of the patterned structural elements in the first image.
Aspect 71. The method of any of Aspects 36-70, the images of the sample are taken by a mobile phone or a consumer camera
Aspect 72. An image-based system for performing the method of any of Aspects 36-71, comprising:
   a device of any of Aspects 1-35;
   a camera module;
   a light source;
   a database system to store images;
   a memory card storing a machine learning model; and
   a processing device, communicatively coupled to the database system.
Aspect 73. An image-based system for performing the method of any of Aspects 36-71, comprising:
   a device of any of Aspects 1-35;
   a mobile phone comprising a camera module and a light source, wherein the mobile phone is capable of communicatively coupled to the database system;
   a non-transitory memory card storing a machine learning model; and
   a database system to store images.

### ASPECT II

1. A method for identifying a cell in a sample, the method comprising:
   obtaining a first plate and a second plate;
   sandwiching the sample between the two plates, where in the first plate and the second plate are facing each other and the spacing between the two plates is less than the dimension of the cell in the direction of the spacing and the cell is compressed in the direction of the spacing;
   imaging one or more images of the cell between the two plates; and
   identifying the cell by analyzing the images.
2. The method of embodiment 1, wherein
   (a) the first plate and the second plate are movable relative to each other and form different configurations, including an open configuration and a closed configuration;
   (b) one or both plate have spacers;
   (c) depositing the sample on one or both plate at the open configuration;
   (d) reducing the spacing between the first and second plates so that the first and second plates are in the closed configuration to compress the sample into a layer whose thickness is less than a dimension of the cell that is not compressed;
   (d) imaging an image of the sample; and
   (e) analyzing the image.
   wherein the open configuration is a configuration in which the first and second plates are either partially or completely separate apart, and the spacing between the first and second plates is not regulated by the spacers; and
   wherein the closed configuration is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the inner surfaces of the two plates and is regulated by the plates and the spacers.
3. The method of embodiment 1 and 2, wherein one or both plates has multiple heights, so that the gap in one area is 2 μm to 10 μm for analyzing neutrophils, lymphocytes, monocytes, eosinophils, and basophils; and the gap in another area of the plate is 20 μm and 40 μm for analyzing total WBC.
4. The method of embodiment 1 and 2, wherein one or both plates has multiple heights, so that the gap in one area is 5 μm for neutrophils, lymphocytes, monocytes, eosinophils, and basophils; and the gap in another area of the plate is 20 μm and 40 μm for analyzing total WBC.
5. The method of embodiment 1 and 2, wherein one or both plates has multiple heights, so that the gap in one area is 2 μm to 10 μm and the gap in another area of the plate is 20 μm and 40 μm.
6. The method of embodiment 1 and 2, wherein one or both plates has multiple heights, so that the gap in one area is 5 μm and the gap in another area of the plate is 30 μm.
7. The method of embodiment 1 and 2, wherein the analysis uses a database generated with a machine learning model to obtain the bio-entity of the sample
2. The method of embodiment 1, wherein the patterned structural element comprises a plurality of spacers affixed at least one of the first and second plates.
3. The method of embodiment 2, wherein the spacers have a height of 1 μm to 150 μm.
4. The method of embodiment 2, wherein the spacers have a rectangle shape with a round corner.
5. The method of embodiment 1, wherein at least one of the first and second plates comprises a sample deposition site.
6. The method of embodiment 5, wherein the sample deposition site is coated with a reagent.
7. The method of embodiment 6, wherein the reagent comprises a staining reagent and/or a detergent.
8. The method of embodiment 7, wherein the detergent is capable of dispersing cells.
9. The method of embodiment 7, wherein the staining reagent comprises a fluorescent stain.
10. The method of embodiment 7, wherein the fluorescent stain comprises at least one selected from the group consisting of acridine orange dye, 3,3-dihexyloxacarbocyanine (DiOC6), Propidium Iodide (PI), Fluorescein Isothiocyanate (FITC) and Basic Orange 21 (BO21) dye, Ethidium Bromide, Brilliant Sulfaflavine and a Stilbene Disulfonic Acid derivative, Erythrosine B or trypan blue, Hoechst 33342, Trihydrochloride, Trihydrate, and DAPI (4′, 6-Diamidino-2-Phenylindole, Dihydrochloride).
11. The method of embodiment 9, wherein the staining agent comprises at least one selected from the group consisting of a Wright's stain, a Giemsa stain, a May-Grunwald stain, a Leishman's stain, and Erythrosine B stain.
12. The method of embodiment 11, wherein Wright's stain comprises at least one selected from the group consisting of Eosin and methylene blue, and the Giemsa stain comprises at least one selected from the group consisting of Eosin, methylene blue, and Azure B.
13. The method of embodiment 7, wherein the detergent comprises at least one selected from a Zwitterionic detergent, an anionic detergent, a cationic Detergent, and a non-ionic detergent.
14. The method of embodiment 13, wherein the anionic detergent comprises at least one selected from the group consisting of Alkyl Sulfates, Alkyl Sulfonates, and Bile Salts.
15. The method of embodiment 7, wherein the reagent comprises acridine orange (AO) and zwittergent 3-14.
16. The method of embodiment 15, wherein the device is a Q-Card.
17. The method of embodiment 1, wherein the sample is a blood sample containing WBC (white blood cells).
18. The method of embodiment 17, wherein the blood sample is a whole blood without dilution.
19. The method of 17, wherein the blood sample is capillary or venous whole blood with or without anti-agglutination as EDTA.
20. The method of 17, wherein step (e) identifies types and amounts of each type of WBC, including as neutrophils, lymphocytes, monocytes, eosinophils, and basophils in the blood sample.
21. The method of embodiment 1, wherein the cell is pressed and deformed in the device to facilitate the imaging and analyzing.
22. The method of embodiment 1, wherein the cell comprises white blood cells, and the white blood cells are pressed to a layer having a thickness of 1 μm, 2 μm, 3 μm, 5 μm, 10 μm, 15 μm or in a range between any two of the thicknesses.
23. The method of embodiment 1, wherein step (e) further comprises:
   extracting cell patches from the image of the sample and feeding the cell patches into a trained deep learning model for cell-type classification.
24. The method of embodiment 1, wherein the image comprises a paired bright-field (BF) image and fluorescence-field (FF) image of the sample.
25. The method of embodiment 23, wherein the image comprises a paired BF image and FF image of the sample, and
   the step of extracting the cell patches further comprises detecting and localizing a cell from the FF image, wherein a key point corresponding to the cell in the sample is detected using a blob detection.
26. The method of embodiment 25, further comprises constructing a bounding box using the key point as a center, wherein the bounding box captures the cell to be detected.
27. The method of embodiment 26, further comprising refining the detection and localization of the cell captured in the image.
28. The method of embodiment 25, wherein the step of extracting the cell patches comprising: collecting an FF image patch around a key point in the image,
   feeding the FF image patch to a second white blood cell classification model to detect if a white blood cell exists in the FF image patch,
   if the FF image patch is classified as containing a white blood cell, a paired BF image patch is constructed from the BF image at the same location so that the FF and BF image patches of the cell are aligned.
29. The method of embodiment 25, wherein the machine learning model is built based on the network structure of DenseNet with input features extracted from cell patches of a paired BF image and FF image of the sample.
30. The method of embodiment 25, further comprising obtaining a matched image pair of a cell from the extracted paired BF image and FF image patches.
31. The method of embodiment 30, further comprising grouping the matched image pair of the cell as an extended vector (BF_cell, FF_cell) for a cell classification/differentiation machine learning model to determine a cell type in the paired image patches.
32. The method of embodiment 31, wherein the matched BF_cell and FF_cell are merged by adding together into one vector to reduce the input dimension for cell classification and differentiation.
33. The method of embodiment 1, wherein a process for building the machine learning model comprises:
   labeling a small number of cells to obtain a first labeled data that is used as a first seed data to generate a first machine learning model for cell classification and differentiation,
   classifying unlabeled data with the first machine learning model to obtain a second labeled data, verifying the second labeled data to obtain a second seed data to train and build a higher quality machine learning model.
34. The method of embodiment 1, further comprising:
   receiving a first image of the device containing the sample;
   identifying a first region in the first image based on locations of one or more structural elements of the patterned structural element in the first image;
   determining a spatial transform associated with the first region based on a mapping between locations of the one or more structural elements in the first image and predetermined positions of the one or more structural elements in the device;
   applying the spatial transform to the first region in the first image to calculate a transformed first region; and
   training the machine learning model using the transformed first image.
35. The method of embodiment 34, wherein the patterned structural element comprises spacers that are distributed periodically with a periodicity value, and wherein detecting the locations of the patterned structural element in the first image includes:
   detecting, using a second machine learning model, the locations of the spacers in the first image; and
   correcting, based on the periodicity value, an error in the detected locations of the spacers in the first image.
36. The method of embodiment 35, wherein the spacers are pillars embedded on at least one of the first plate or the second plate.
37. The method of embodiment 35, further comprising:
   detecting locations of the patterned structural element of the device in a first image;
   partitioning the first image into regions comprising a first region, wherein each of the regions is defined by four structural elements at four corners of the corresponding region;
   determining a corresponding spatial transform associated with each of the regions in the first image based on a mapping between the locations of the four structural elements at the four corners of the corresponding region and the four predetermined positions of the four structural elements in the device;
   applying the corresponding spatial transform to each of the regions in the first image to calculate a corresponding transformed region in the first image; and
   training the machine learning model using each of transformed regions in the first image to obtain a trained machine learning model,
   wherein the trained machine learning model is used to detect cells from the image of the sample and differentiate them into different cell classes based on the transformed image.
38. The method of embodiment 1, further comprising:
   receiving a first image of the device containing the sample;
   identifying a first region in the first image based on locations of one or more structural elements of the patterned structural elements in the first image;
   determining a spatial transform associated with the first region based on a mapping between the locations of the one or more structural elements in the first image and predetermined positions of one or more structural elements in device;
   applying the spatial transform to the first region in the first image to calculate a transformed first region;
   training the machine learning model based on the transformed first region, and applying the machine learning model to detect and differentiate cells from the transformed region of the image of the sample.
39. The method of embodiment 38, further comprising:
   partitioning the first image into a plurality of regions based the locations of the one or more structural elements of the patterned structural elements in the first image, wherein the plurality of regions comprises the first region;
   determining a respective spatial transform associated with each of the plurality of regions; applying the corresponding spatial transform to each of the plurality of regions in the first image to calculate transformed regions;
   applying the machine learning model to each of the transformed regions in the first image to perform cell detection and differentiation.
40. The method of embodiment 1, the image of the sample is taken by a mobile phone or a consumer camera.
41. The method of embodiment 1, wherein step (e) is conducted with an image-based assay system comprising:
   a database system to store images; and
   a processing device, communicatively coupled to the database system, to:
   receive a first image of the sample in the device, wherein the patterned structural element comprising one or more structural elements having predetermined positions;
   identify a first region in the first image based on locations of the one or more structural elements in the first image;
   determine a spatial transform associated with the first region based on a mapping between the locations of the one or more structural elements in the first image and predetermined positions of the one or more structural elements in device;
   apply the spatial transform to the first region in the first image to calculate a transformed first region; and
   train a machine learning model using the transformed first image.
42. The method of embodiment 41, wherein the one or more structural elements are pillars embedded at predetermined positions on at least one of the first plate and the second plate.
43. The method of embodiment 42, wherein the processing device is further to: detect the locations of the patterned structural element in the first image;
   partition the first image into regions comprising the first region, wherein each of the regions is defined by four structural elements at four corners of the corresponding region;
   determine a corresponding spatial transform associated with each of the regions in the first image based on a mapping between the locations of the four structural elements at the four corners of the corresponding region and the four predetermined positions of the four structural elements in the device;
   apply the corresponding spatial transform to each of the regions in the first image to calculate a corresponding transformed region in the first image; and
   train the machine learning model using each of transformed regions in the first image, wherein the trained machine learning model is used to do cell detection and differentiation.
44. The method of embodiment 43, wherein positions of the patterned structural element are distributed periodically with at least one periodicity value, and
   Wherein, to detect the locations of the patterned structural elements in the first image, the processing device is further to:
   detect, using a second machine learning model, the locations of the patterned structural elements in the first image; and
   correct, based on the at least one periodicity value, an error in the detected locations of the patterned structural elements in the first image

The disclosure provides a method for identifying a cell in a sample, the method includes:

obtaining a sample holder including a first plate and a second plate; sandwiching the sample between the two plates; pressing the places to decrease the spacing therebetween to a dimension smaller than a size of the cell that is not compressed by the plates; imaging one or more images of the cell compressed between the two plates; and identifying the cell by analyzing the one or more images.

In an embodiment, the first plate and the second plate are movable relative to each other and form different configurations, including an open configuration and a closed configuration, one or both plates include spacers, the sample is deposited on one or both plates at the open configuration, and the pressing step brings the first and second plates into the closed configuration, which compresses the sample into a layer whose thickness is smaller than a size of the cell that is uncompressed.

In an embodiment, the open configuration is a configuration in which the first and second plates are either partially or completely separate apart, and the spacing between the first and second plates is not regulated by the spacers; and

In an embodiment, the closed configuration is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the inner surfaces of the two plates and is regulated by the plates and the spacers.

In an embodiment, the one or both plates have multiple heights to provide areas have different gaps between the two plates in the closed configuration, one of the areas have a gap in a range of 2 μm to 10 μm for analyzing neutrophils, lymphocytes, monocytes, eosinophils, and basophils; and another area has a gap in a range of 20 μm to 40 μm for analyzing total WBC.

In an embodiment, the one or both plates have multiple heights to provide areas have different gaps between the two plates in the closed configuration, one of the areas have a gap of 5 μm for neutrophils, lymphocytes, monocytes, eosinophils, and basophils; and another area has a gap in a range of 20 μm to 40 μm for analyzing total WBC.

In an embodiment, the one or both plates have multiple heights to provide areas have different gaps between the two plates in the closed configuration, one of the areas have a gap of 5 μm, and another area has a gap of 30 μm.

In an embodiment, the analyzing uses a database generated with a machine learning model to obtain the bio-entity of the sample.

In an embodiment, the sample holder includes a patterned structural element affixed to at least one of the first and second plates. In an embodiment, the patterned structural elements include spacers. In an embodiment, the spacers have a rectangle shape with a round corner or a pillar shape.

In an embodiment, at least one of the first and second plates includes a sample deposition site. In an embodiment, the sample deposition site is coated with a reagent. In an embodiment, the reagent includes a staining reagent and/or a detergent. In an embodiment, the staining agent includes at least one selected from the group consisting of a Wright's stain, a Giemsa stain, a May-Grunwald stain, a Leishman's stain, and Erythrosine B stain. In an embodiment, Wright's stain includes at least one selected from the group consisting of Eosin and methylene blue, and the Giemsa stain includes at least one selected from the group consisting of Eosin, methylene blue, and Azure B. In an embodiment, the detergent is capable of dispersing cells. In an embodiment, the detergent comprises at least one selected from a Zwitterionic detergent, an anionic detergent, a cationic Detergent, and a non-ionic detergent. In an embodiment, the anionic detergent comprises at least one selected from the group consisting of Alkyl Sulfates, Alkyl Sulfonates, and Bile Salts. In an embodiment, the staining reagent comprises a fluorescent stain. In an embodiment, the fluorescent stain comprises at least one selected from the group consisting of acridine orange dye, 3,3-dihexyloxacarbocyanine (DiOC6), Propidium Iodide (PI), Fluorescein Isothiocyanate (FITC) and Basic Orange 21 (BO21) dye, Ethidium Bromide, Brilliant Sulfaflavine and a Stilbene Disulfonic Acid derivative, Erythrosine B or trypan blue, Hoechst 33342, Trihydrochloride, Trihydrate, and DAPI (4′,6-Diamidino-2-Phenylindole, Dihydrochloride). In an embodiment, wherein the reagent includes acridine orange (AO) and zwittergent 3-14.

In an embodiment, the sample holder is a Q-Card. In an embodiment, the sample is a blood sample containing WBC (white blood cells). In an embodiment, the blood sample is whole blood without dilution. In an embodiment, the blood sample is capillary or venous whole blood with or without anti-agglutination as EDTA. In an embodiment, the sample is a blood sample, and the identifying step includes identifying types and amounts of each type of WBC including neutrophils, lymphocytes, monocytes, eosinophils, and basophils in the blood sample. In an embodiment, the cell is compressed and deformed in the sample holder to facilitate the imaging and analyzing. In an embodiment, the cell includes white blood cells, and the white blood cells are compressed to a layer having a thickness of 1 μm, 2 μm, 3 μm, 5 μm, 10 μm, 15 μm or in a range between any two of the thicknesses.

In an embodiment, the method further includes measuring and analyzing the image against a database generated with a machine learning model to obtain the bio-entity of the sample. In an embodiment, the method further includes extracting cell patches from the image of the sample and feeding the cell patches into a trained deep learning model for cell-type classification. In an embodiment, the image includes a paired bright-field (BF) image and fluorescence-field (FF) image of the sample. In an embodiment, the image includes a paired BF image and FF image of the sample, and the step of extracting the cell patches further includes detecting and localizing a cell from the FF image, in which a key point corresponding to the cell in the sample is detected using a blob detection. In an embodiment, the method further includes constructing a bounding box using the key point as a center, wherein the bounding box captures the cell to be detected. In an embodiment, the method further includes refining the detection and localization of the cell captured in the image.

In an embodiment, the step of extracting the cell patches includes: collecting an FF image patch around a key point in the image; feeding the FF image patch to a second white blood cell classification model to detect if a white blood cell exists in the FF image patch; if the FF image patch is classified as containing a white blood cell, a paired BF image patch is constructed from the BF image at the same location so that the FF and BF image patches of the cell are aligned.

In an embodiment, the machine learning model is built based on the network structure of DenseNet with input features extracted from cell patches of a paired BF image and FF image of the sample. In an embodiment, the method further includes obtaining a matched image pair of a cell from the extracted paired BF image and FF image patches. In an embodiment, the method further includes grouping the matched image pair of the cell as an extended vector (BF_cell, FF_cell) for a cell classification/differentiation machine learning model to determine a cell type in the paired image patches. In an embodiment, the matched BF_cell and FF_cell are merged by adding together into one vector to reduce the input dimension for cell classification and differentiation.

In an embodiment, a process for building the machine learning model includes: labeling a small number of cells to obtain a first labeled data that is used as a first seed data to generate a first machine learning model for cell classification and differentiation; classifying unlabeled data with the first machine learning model to obtain a second labeled data; and verifying the second labeled data to obtain a second seed data to train and build a higher quality machine learning model.

In an embodiment, the method further includes: receiving a first image of the device containing the sample; identifying a first region in the first image based on locations of one or more structural elements of the patterned structural element in the first image; determining a spatial transform associated with the first region based on a mapping between locations of the one or more structural elements in the first image and predetermined positions of the one or more structural elements in the device; applying the spatial transform to the first region in the first image to calculate a transformed first region; and training the machine learning model using the transformed first image. In an embodiment, the patterned structural element includes spacers that are distributed periodically with a periodicity value. In an embodiment, detecting the locations of the patterned structural element in the first image includes: detecting, using a second machine learning model, the locations of the spacers in the first image; and correcting, based on the periodicity value, an error in the detected locations of the spacers in the first image. In an embodiment, the spacers are pillars embedded on at least one of the first plate or the second plate.

In an embodiment, the method further includes: detecting locations of the patterned structural element of the device in a first image; partitioning the first image into regions including a first region, wherein each of the regions is defined by four structural elements at four corners of the corresponding region; determining a corresponding spatial transform associated with each of the regions in the first image based on a mapping between the locations of the four structural elements at the four corners of the corresponding region and the four predetermined positions of the four structural elements in the device; applying the corresponding spatial transform to each of the regions in the first image to calculate a corresponding transformed region in the first image; and training the machine learning model using each of transformed regions in the first image to obtain a trained machine learning model. In an embodiment, the trained machine learning model is used to detect cells from the image of the sample and differentiate them into different cell classes based on the transformed image.

In an embodiment, the method further includes: receiving a first image of the device containing the sample; identifying a first region in the first image based on locations of one or more structural elements of the patterned structural elements in the first image; determining a spatial transform associated with the first region based on a mapping between the locations of the one or more structural elements in the first image and predetermined positions of one or more structural elements in device; applying the spatial transform to the first region in the first image to calculate a transformed first region; training the machine learning model based on the transformed first region, and applying the machine learning model to detect and differentiate cells from the transformed region of the image of the sample. In an embodiment, the method further includes: partitioning the first image into a plurality of regions based the locations of the one or more structural elements of the patterned structural elements in the first image, wherein the plurality of regions includes the first region; determining a respective spatial transform associated with each of the plurality of regions; applying the corresponding spatial transform to each of the plurality of regions in the first image to calculate transformed regions; applying the machine learning model to each of the transformed regions in the first image to perform cell detection and differentiation.

In an embodiment, the image of the sample is taken by a mobile phone or a consumer camera.

In an embodiment, the step of measuring and analyzing the image is conducted with an image-based assay system includes: a database system to store images; and a processing device, which is communicatively coupled to the database system to: receive a first image of the sample in the device, wherein the patterned structural element including one or more structural elements having predetermined positions; identify a first region in the first image based on locations of the one or more structural elements in the first image; determine a spatial transform associated with the first region based on a mapping between the locations of the one or more structural elements in the first image and predetermined positions of the one or more structural elements in device; apply the spatial transform to the first region in the first image to calculate a transformed first region; and train a machine learning model using the transformed first image. In an embodiment, the one or more structural elements are pillars embedded at predetermined positions on at least one of the first plate and the second plate. In an embodiment, the processing device is further to: detect the locations of the patterned structural element in the first image; partition the first image into regions including the first region, wherein each of the regions is defined by four structural elements at four corners of the corresponding region; determine a corresponding spatial transform associated with each of the regions in the first image based on a mapping between the locations of the four structural elements at the four corners of the corresponding region and the four predetermined positions of the four structural elements in the device; apply the corresponding spatial transform to each of the regions in the first image to calculate a corresponding transformed region in the first image; and train the machine learning model using each of transformed regions in the first image, wherein the trained machine learning model is used to do cell detection and differentiation.

In an embodiment, the predetermined positions of the patterned structural element are distributed periodically with at least one periodicity value. In an embodiment, to detect the locations of the patterned structural elements in the first image, the processing device is further configured to: detect, using a second machine learning model, the locations of the patterned structural elements in the first image; and correct, based on the at least one periodicity value, an error in the detected locations of the patterned structural elements in the first image.

### ASPECTS

1. A method for identifying a cell in a sample, the method comprising:
   obtaining a sample holder comprising a first plate and a second plate;
   sandwiching the sample between the two plates;
   pressing the places to decrease the spacing therebetween to a dimension smaller than a size of the cell that is not compressed by the plates;
   imaging one or more images of the cell compressed between the two plates; and
   identifying the cell by analyzing the one or more images.
2. The method of Aspect 1, wherein
   (e) the first plate and the second plate are movable relative to each other and form different configurations, including an open configuration and a closed configuration;
   (f) one or both plates comprise spacers;
   (g) the sample is deposited on one or both plates at the open configuration; and
   (h) the pressing step brings the first and second plates into the closed configuration, which compresses the sample into a layer whose thickness is smaller than a size of the cell that is uncompressed,
   wherein the open configuration is a configuration in which the first and second plates are either partially or completely separate apart, and the spacing between the first and second plates is not regulated by the spacers; and
   wherein the closed configuration is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the inner surfaces of the two plates and is regulated by the plates and the spacers.
3. The method of Aspect 1, wherein one or both plates have multiple heights to provide areas have different gaps between the two plates in the closed configuration, one of the areas have a gap in a range of 2 μm to 10 μm for analyzing neutrophils, lymphocytes, monocytes, eosinophils, and basophils; and another area has a gap in a range of 20 μm to 40 μm for analyzing total WBC.
4. The method of Aspect 1, wherein one or both plates have multiple heights to provide areas have different gaps between the two plates in the closed configuration, one of the areas have a gap of 5 μm for neutrophils, lymphocytes, monocytes, eosinophils, and basophils; and another area has a gap in a range of 20 μm to 40 μm for analyzing total WBC.
5. The method of Aspect 3, wherein one or both plates have multiple heights to provide areas have different gaps between the two plates in the closed configuration, one of the areas have a gap of 5 μm, and another area has a gap of 30 μm.
6. The method of Aspect 1, wherein the analyzing uses a database generated with a machine learning model to obtain the bio-entity of the sample.
7. The method of embodiment 1, wherein the sample holder comprises a patterned structural element affixed to at least one of the first and second plates.
8. The method of Aspect 7, wherein the patterned structural elements comprise spacers.
9. The method of Aspect 8, wherein the spacers have a rectangle shape with a round corner or a pillar shape.
10. The method of Aspect 1, wherein at least one of the first and second plates comprises a sample deposition site.
11. The method of Aspect 10, wherein the sample deposition site is coated with a reagent.
12. The method of Aspect 11, wherein the reagent comprises a staining reagent and/or a detergent.
13. The method of Aspect 12, wherein the staining agent comprises at least one selected from the group consisting of a Wright's stain, a Giemsa stain, a May-Grunwald stain, a Leishman's stain, and Erythrosine B stain.
14. The method of Aspect 12, wherein Wright's stain comprises at least one selected from the group consisting of Eosin and methylene blue, and the Giemsa stain comprises at least one selected from the group consisting of Eosin, methylene blue, and Azure B.
15. The method of Aspect 12, wherein the detergent is capable of dispersing cells.
16. The method of Aspect 15, wherein the detergent comprises at least one selected from a Zwitterionic detergent, an anionic detergent, a cationic Detergent, and a non-ionic detergent.
17. The method of embodiment 16, wherein the anionic detergent comprises at least one selected from the group consisting of Alkyl Sulfates, Alkyl Sulfonates, and Bile Salts.
18. The method of Aspect 12, wherein the staining reagent comprises a fluorescent stain.
19. The method of Aspect 14, wherein the fluorescent stain comprises at least one selected from the group consisting of acridine orange dye, 3,3-dihexyloxacarbocyanine (DiOC6), Propidium Iodide (PI), Fluorescein Isothiocyanate (FITC) and Basic Orange 21 (BO21) dye, Ethidium Bromide, Brilliant Sulfaflavine and a Stilbene Disulfonic Acid derivative, Erythrosine B or trypan blue, Hoechst 33342, Trihydrochloride, Trihydrate, and DAPI (4′, 6-Diamidino-2-Phenylindole, Dihydrochloride).
20. The method of Aspect 11, wherein the reagent comprises acridine orange (AO) and zwittergent 3-14.
21. The method of Aspect 1, wherein the sample holder is a Q-Card.
22. The method of Aspect 1, wherein the sample is a blood sample containing WBC (white blood cells).
23. The method of Aspect 22, wherein the blood sample is whole blood without dilution.
24. The method of Aspect 22, wherein the blood sample is capillary or venous whole blood with or without anti-agglutination as EDTA.
25. The method of Aspect 1, wherein the sample is a blood sample, and the identifying step includes identifying types and amounts of each type of WBC including neutrophils, lymphocytes, monocytes, eosinophils, and basophils in the blood sample.
26. The method of Aspect 1, wherein the cell is compressed and deformed in the sample holder to facilitate the imaging and analyzing.
27. The method of embodiment 1, wherein the cell comprises white blood cells, and the white blood cells are compressed to a layer having a thickness of 1 μm, 2 μm, 3 μm, 5 μm, 10 μm, 15 μm or in a range between any two of the thicknesses.
28. The method of Aspect 1, further comprising measuring and analyzing the image against a database generated with a machine learning model to obtain the bio-entity of the sample.
29. The method of Aspect 28, further comprising:
   extracting cell patches from the image of the sample and feeding the cell patches into a trained deep learning model for cell-type classification.
30. The method of Aspect 29, wherein the image comprises a paired bright-field (BF) image and fluorescence-field (FF) image of the sample.
31. The method of Aspect 29, wherein the image comprises a paired BF image and FF image of the sample, and the step of extracting the cell patches further comprises detecting and localizing a cell from the FF image, wherein a key point corresponding to the cell in the sample is detected using a blob detection.
32. The method of Aspect 29, further comprises constructing a bounding box using the key point as a center, wherein the bounding box captures the cell to be detected.
33. The method of Aspect 29, further comprising refining the detection and localization of the cell captured in the image.
34. The method of embodiment 29, wherein the step of extracting the cell patches comprising:
   a. collecting an FF image patch around a key point in the image,
   b. feeding the FF image patch to a second white blood cell classification model to detect if a white blood cell exists in the FF image patch,
   c. if the FF image patch is classified as containing a white blood cell, a paired BF image patch is constructed from the BF image at the same location so that the FF and BF image patches of the cell are aligned.
35. The method of Aspect 28, wherein the machine learning model is built based on the network structure of DenseNet with input features extracted from cell patches of a paired BF image and FF image of the sample.
36. The method of Aspect 29, further comprising obtaining a matched image pair of a cell from the extracted paired BF image and FF image patches.
37. The method of Aspect 36, further comprising grouping the matched image pair of the cell as an extended vector (BF_cell, FF_cell) for a cell classification/differentiation machine learning model to determine a cell type in the paired image patches.
38. The method of Aspect 37, wherein the matched BF_cell and FF_cell are merged by adding together into one vector to reduce the input dimension for cell classification and differentiation.
39. The method of Aspect 28, wherein a process for building the machine learning model comprises:
   labeling a small number of cells to obtain a first labeled data that is used as a first seed data to generate a first machine learning model for cell classification and differentiation,
   classifying unlabeled data with the first machine learning model to obtain a second labeled data, and
   verifying the second labeled data to obtain a second seed data to train and build a higher quality machine learning model.
40. The method of Aspect 7, further comprising:
   receiving a first image of the device containing the sample;
   identifying a first region in the first image based on locations of one or more structural elements of the patterned structural element in the first image;
   determining a spatial transform associated with the first region based on a mapping between locations of the one or more structural elements in the first image and predetermined positions of the one or more structural elements in the device;
   applying the spatial transform to the first region in the first image to calculate a transformed first region; and
   training the machine learning model using the transformed first image.
41. The method of Aspect 40, wherein the patterned structural element comprises spacers that are distributed periodically with a periodicity value, and wherein detecting the locations of the patterned structural element in the first image includes:
   detecting, using a second machine learning model, the locations of the spacers in the first image; and
   correcting, based on the periodicity value, an error in the detected locations of the spacers in the first image.
42. The method of Aspect 41, wherein the spacers are pillars embedded on at least one of the first plate or the second plate.
43. The method of Aspect 41, further comprising:
   detecting locations of the patterned structural element of the device in a first image;
   partitioning the first image into regions comprising a first region, wherein each of the regions is defined by four structural elements at four corners of the corresponding region;
   determining a corresponding spatial transform associated with each of the regions in the first image based on a mapping between the locations of the four structural elements at the four corners of the corresponding region and the four predetermined positions of the four structural elements in the device;
   applying the corresponding spatial transform to each of the regions in the first image to calculate a corresponding transformed region in the first image; and
   training the machine learning model using each of transformed regions in the first image to obtain a trained machine learning model,
   wherein the trained machine learning model is used to detect cells from the image of the sample and differentiate them into different cell classes based on the transformed image.
44. The method of Aspect 1, further comprising:
   receiving a first image of the device containing the sample;
   identifying a first region in the first image based on locations of one or more structural elements of the patterned structural elements in the first image;
   determining a spatial transform associated with the first region based on a mapping between the locations of the one or more structural elements in the first image and predetermined positions of one or more structural elements in device;
   applying the spatial transform to the first region in the first image to calculate a transformed first region;
   training the machine learning model based on the transformed first region, and
   applying the machine learning model to detect and differentiate cells from the transformed region of the image of the sample.
45. The method of embodiment 44, further comprising:
   partitioning the first image into a plurality of regions based the locations of the one or more structural elements of the patterned structural elements in the first image, wherein the plurality of regions comprises the first region;
   determining a respective spatial transform associated with each of the plurality of regions;
   applying the corresponding spatial transform to each of the plurality of regions in the first image to calculate transformed regions;
   applying the machine learning model to each of the transformed regions in the first image to perform cell detection and differentiation.
46. The method of Aspect 1, the image of the sample is taken by a mobile phone or a consumer camera.
47. The method of Aspect 28, wherein the step of measuring and analyzing the image is conducted with an image-based assay system comprising:
   a database system to store images; and
   a processing device, communicatively coupled to the database system, to:
      receive a first image of the sample in the device, wherein the patterned structural element comprising one or more structural elements having predetermined positions;
      identify a first region in the first image based on locations of the one or more structural elements in the first image;
      determine a spatial transform associated with the first region based on a mapping between the locations of the one or more structural elements in the first image and predetermined positions of the one or more structural elements in device;
      apply the spatial transform to the first region in the first image to calculate a transformed first region; and
      train a machine learning model using the transformed first image.
48. The method of Aspect 47, wherein the one or more structural elements are pillars embedded at predetermined positions on at least one of the first plate and the second plate.
49. The method of Aspect 47, wherein the processing device is further to:
   detect the locations of the patterned structural element in the first image;
   partition the first image into regions comprising the first region, wherein each of the regions is defined by four structural elements at four corners of the corresponding region;
   determine a corresponding spatial transform associated with each of the regions in the first image based on a mapping between the locations of the four structural elements at the four corners of the corresponding region and the four predetermined positions of the four structural elements in the device;
   apply the corresponding spatial transform to each of the regions in the first image to calculate a corresponding transformed region in the first image; and
   train the machine learning model using each of transformed regions in the first image, wherein the trained machine learning model is used to do cell detection and differentiation.
50. The method of Aspect 49, wherein the predetermined positions of the patterned structural element are distributed periodically with at least one periodicity value, and wherein, to detect the locations of the patterned structural elements in the first image, the processing device is further configured to:
   detect, using a second machine learning model, the locations of the patterned structural elements in the first image; and
   correct, based on the at least one periodicity value, an error in the detected locations of the patterned structural elements in the first image.
51. The method of Aspect 1, further comprising: pressing the places to decrease the spacing therebetween to a dimension smaller than a size of the cell that is not compressed by the plates.
52. The method of Aspect 2, wherein the image includes an image of the spacers, and wherein the spacers function as scale marker for training the machine learning model. (there are many machine learning claims in this list, and you can try searching “machine learning”.)
53. The method of claim 1, the identifying of the cell and the analyzing of images uses a machine learning model.
54. The method of Aspect 2, wherein the spaces have an inter-space-distance (ISD); at least one of the plates is flexible;
   for the flexible plate, a fourth power of the inter-spacer-distance (ISD) divided by the thickness of the flexible plate (h) and a Young's modulus (E) of the flexible plate, ISD⁴/(hE), is equal to or less than 10⁶ μm³/GPa;
   the thickness of the flexible plate times the Young's modulus of the flexible plate is in the range of 60 to 750 GPa-μm.
55. The method of Aspect 2, wherein the cell is white blood cell, and the two plates in the closed configuration have a spacing in a range 3 μm to 6 μm for analyzing white blood cell differentials.
56. The method of Aspect 2, wherein the cell is white blood cell, and the two plates in the closed configuration have a spacing of 5 μm for analyzing white blood cell differentials.

We claim:

1. A method for identifying a cell in a sample, the method comprising:
obtaining a sample holder comprising a first plate and a second plate;
sandwiching the sample between the two plates; where in the first plate and the second plate are facing each other and the spacing between the two plates is less than the dimension of the cell in the direction of the spacing and the cell is compressed in the direction of the spacing
imaging one or more images of the cell compressed between the two plates; and
analyzing the cell by analyzing the one or more images.

2. The method of claim 1, wherein
(a) the first plate and the second plate are movable relative to each other and form different configurations, including an open configuration and a closed configuration;
(b) one or both plates comprise spacers;
(c) the sample is deposited on one or both plates at the open configuration; and
(d) the pressing step brings the first and second plates into the closed configuration, which compresses the sample into a layer whose thickness is smaller than a size of the cell that is uncompressed,
wherein the open configuration is a configuration in which the first and second plates are either partially or completely separate apart, and the spacing between the first and second plates is not regulated by the spacers; and
wherein the closed configuration is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the inner surfaces of the two plates and is regulated by the plates and the spacers.

3. The method of claim 1, wherein the sample holder comprises the spacers that regulate the spacing between the first plate and the second plate.

4. The method of claim 1 further comprising a step of pressing the places, after the cell sandwich between the two plates, to decrease the spacing to a dimension smaller than a size of the cell that is not compressed by the plates.

5. The method of claim 3 or claim 3, wherein the image includes an image of the spacers, and wherein the spacers function as scale marker for training the machine learning model. (there are many machine learning claims in this list, and you can try searching “machine learning”.)

6. The method of claim 1 or 2, the identifying of the cell and the analyzing of images uses a machine learning model.

7. The method of claim 2 or 3, wherein the machine learning model uses an image with the spacers in analyzing the cell in the sample.

8. The method of claim 2, wherein the spaces have an inter-space-distance (ISD); at least one of the plates is flexible;
for the flexible plate, a fourth power of the inter-spacer-distance (ISD) divided by the thickness of the flexible plate (h) and a Young's modulus (E) of the flexible plate, ISD⁴/(hE), is equal to or less than 10⁶ μm³/GPa;
the thickness of the flexible plate times the Young's modulus of the flexible plate is in the range of 60 to 750 GPa-μm.

9. The method of claim 1 or 2, wherein the cell is white blood cell, the two plates in the closed configuration have a spacing in a range 3-6 μm, and the analyzing is to identify the differentials of the white blood cell (i.e. neutrophils, lymphocytes, monocytes, eosinophils, and basophils)

10. The method of claim 2, wherein the cell is white blood cell, and the two plates in the closed configuration have a spacing of 5 μm for analyzing white blood cell differentials (neutrophils, lymphocytes, monocytes, eosinophils, and basophils).

11. The method of claim 1 or claim 2, wherein one or both plates have multiple heights to provide areas have different gaps between the two plates in the closed configuration, one of the areas have a gap in a range of 2 μm to 10 μm for analyzing neutrophils, lymphocytes, monocytes, eosinophils, and basophils; and another area has a gap in a range of 20 μm to 40 μm for analyzing total WBC.

12. The method of claim 1 or claim 2, wherein one or both plates have multiple heights to provide areas have different gaps between the two plates in the closed configuration, one of the areas have a gap of 5 μm for neutrophils, lymphocytes, monocytes, eosinophils, and basophils; and another area has a gap in a range of 20 μm to 40 μm for analyzing total WBC.

13. The method of claim 1, 2 or 3, wherein one or both plates have multiple heights to provide areas have different gaps between the two plates in the closed configuration, one of the areas have a gap of 5 μm, and another area has a gap of 30 μm.

14. The method of claim 1 or claim 2 or claim 3, wherein at least one of the first and second plates comprises a sample deposition site.

15. The method of claim 10, wherein the sample deposition site is coated with a reagent.

16. The method of claim 11, wherein the reagent comprises a staining reagent and/or a detergent.

17. The method of claim 12, wherein the staining agent comprises at least one selected from the group consisting of a Wright's stain, a Giemsa stain, a May-Grunwald stain, a Leishman's stain, and Erythrosine B stain.

18. The method of claim 15, wherein the detergent comprises at least one selected from a Zwitterionic detergent, an anionic detergent, a cationic Detergent, and a non-ionic detergent.

19. The method of claim 11, wherein the reagent comprises acridine orange (AO) and zwittergent 3-14.

20. The method of claim 1, claim 2, or claim 3, wherein the cell in the sample is a blood sample containing WBC (white blood cells).

21. The method of claim 1, further comprising measuring and analyzing the image against a database generated with a machine learning model to obtain the bio-entity of the sample, and wherein the image contains an image of the spacers.

22. The method of claim 17, wherein a process for building the machine learning model comprises:
labeling a small number of cells to obtain a first labeled data that is used as a first seed data to generate a first machine learning model for cell classification and differentiation,
classifying unlabeled data with the first machine learning model to obtain a second labeled data, and
verifying the second labeled data to obtain a second seed data to train and build a higher quality machine learning model.
